(19) United States
(12) Patent Application Publication
(10) Pub. No.: US 2009/0124639 A1
(43) Pub. Date: May 14, 2009

(54) VALACYCLOVIR FORMULATIONS

(75) Inventors: Moses Oyewumi, Yorktown Heights, NY (US); Shingai Majuru, Brewster, NY (US); Lewis Bender, Redding, CT (US); Akm Nasir Uddin, West Harrison, NY (US)

Correspondence Address: Emisphere Technologies, Inc, 240 Cedar Knolls Road, Suite # 200, Cedar Knolls, NJ, 07927, US

(73) Assignee: Emisphere Technologies Inc., Cedar Knolls, NJ (US)

(21) Appl. No.: 12/266,510

(22) Filed: Nov. 6, 2008

Related U.S. Application Data
(60) Provisional application No. 60/985,767, filed on Nov. 6, 2007.

(51) Int. Cl. A61K 31/522; A61P 31/22

(52) U.S. Cl. 514/263.38

(57) ABSTRACT

The present invention relates to valacyclovir formulations having improved bioavailability resulting in better efficacy and/or requiring less frequent administration.

### FIELD OF THE INVENTION

The invention describes valacyclovir dosage forms (e.g., orally dosed tablets and capsules) containing a delivery agent compound resulting in improved relative valacyclovir bioavailability.

### BACKGROUND OF THE INVENTION

Valacyclovir is the L-valine ester of acyclovir, an antiviral agent that is the prodrug of acyclovir and is active against herpes viruses. Oral valacyclovir is used for the treatment of initial and recurrent episodes of genital herpes infections in immunocompetent adults and adolescents and for the suppression of recurrent episodes of genital herpes in immunocompetent adults and adolescents and individuals infected with human immunodefficency virus (HIV). Oral valacyclovir may also be used for the episodic treatment of herpes labialis (cold sores) in adults and adolescents and for the treatment of acute, localized herpes zoster (shingles, zoster) in adults and adolescents.

Acyclovir (9-((2-hydroxyethoxy)methyl)guanine) is an antiviral which inhibits human herpes viruses, including herpes simplex types 1 (HSV-1) and 2 (1HSV-2), varicella zoster, Epstein-Barr virus (EBV) and cytomegalovirus (CMV). The inhibitory activity of acyclovir is highly selective for these viruses. O'Brien and Campoli-Richards, Drugs, 37:233-309 (1989). The chemical composition of acyclovir is reported in Shaffer, et al. (J. Med. Chem. 14:367 (1971)), U.S. Pat. No. 4,199,574, and UK Patent Specification No. 1,523,865, all of which are hereby incorporated by reference.

Acyclovir has been demonstrated to be a potent antiviral agent, particularly against herpes viruses. Shaffer, et al. Nature 272:583-585 (1978). Acyclovir has also been demonstrated to effectively suppress reactivated or newly acquired viral diseases such as genital herpes simplex, shingles, and varicella-zoster virus (VZV), as well as acute varicelta-zoster infections. Balfour, J. Med Virology, S1:74-81 (1993). Morbidity and mortality from viral disease have been reduced by pre- and postoperative prophylaxis with long-term (>6 months) oral acyclovir therapy. Prentice et al., Lancet 343:749-753 (1994). Concurrent acyclovir and AZT (azidothymidine) therapy has extended the survival of AIDS patients by one year when acyclovir therapy was begun at time of diagnosis. Stein, et al., Ann. Intern. Med. 121:100-108 (1994). Additionally, acyclovir therapy for acute varicella-zoster disease reduces fever, clionic pain, and the progression of rash and accelerates cutaneous healing.

Other uses of aceyclovir include, but are not limited to, mucocutanieous, ocular, and systemic herpes simplex infections (HSV), including in human immunodeficiency virus (HIV)-infected individuals. It is also useful to treat HSV encephalitis, neonatal HSV infections, and genital herpes (first episode, recurrent and suppressive therapy for recurrent infections). Further, acyclovir is effective therapy for varicella-zoster infections, herpes zoster (shingles, zoster), cytomegalovirus infections, infections and disorders associated with Epstein-Barr virus, and the Center for Disease Control states that oral acyclovir may be used in pregnant women. These and other uses are found in AHFS Drug Information, American Society of Health System Pharmacists, Bethesda, Md., 2005, which is incorporated by reference herein.

U.S. Pat. No. 5,629,016, which is hereby incorporated by reference, discloses water dispersible tablets containing acyclovir which facilitates the ingestion of large doses (i.e. up to 800 mg) of acyclovir.

U.S. Pat. No. 5,883,103 discloses a microemulsion system for the oral delivery of acyclovir. The system includes a water-in-oil emulsion with acyclovir dispersed in aqueous phase droplets. The droplets have an average droplet size of 20-40 nanometers and are uniformly dispersed in the continuous oil phase.

Valacyclovir is a prodrug and exhibits no activity until it is hydrolyzed in the intestinal wall and/or liver and converted to acyclovir, and its active metabolite, acyclovir triphosphate. Valacyclovir hydrochloride (commercially available as Valtrex®) differs structurally from acyclovir by having the 1-amino acid, valine, attached at the 5′ hydroxyl group of the nucleoside and by the presence of the monohydride salt. These modifications provide an increase in plasma acyclovir concentrations, compared to acyclovir. While acyclovir is poorly absorbed from the GI tract, valacyclovir is converted to acyclovir in vivo and subsequently to the pharmacologically active triphosphate metabolite which has activity against HSV types 1 and 2, VZV, CMV, and EBV.

Valtrex® is available from GlaxoSmithKline in both 500 mg and 1 gram dosage caplets. Each caplet contains valacyclovir hydrochloride equivalent to 500 mg or 1 gram valacyclovir, respectively. The inactive ingredients include carnauba wax, colloidal silicon dioxide, crospovidone, FD&C Blue No. 2 Lake, hypromellose, magnesium stearate, microcrystalline cellulose, polyethylene glycol, polysorbate 80, povidone, and titanium dioxide. The blue, film-coated caplets are printed with edible white ink.

The FDA Orangebook entry for Valtrex® (valacyclovir hydrochloride) lists three U.S. Patents. U.S. Pat. No. 4,957,924 to Beauchamp discloses therapeutic valine esters of acyclovir, U.S. Pat. No. 5,879,706 to Carter et al. discloses valacyclovir tablets containing colloidal silicon dioxide, and U.S. Pat. No. 6,107,302 to Canter et al. discloses an anhydrous crystalline form of valacyclovir hydrochloride. All three patents are hereby incorporated by reference in their entirety.

Although, previous attempts have been made to improve the delivery and bioavailability of valacyclovir, these attempts have had limited success. Therefore, there is a need for oral valacyclovir formulations having increased valacyclovir bioavailability and/or require less frequent dosing when administered to a human.

### SUMMARY OF THE INVENTION

The present invention provides a composition (e.g., a pharmaceutical composition) comprising (a) at least one delivery agent compound and (b) valacyclovir or a salt, ester, or prodrug thereof. Preferably, the composition includes a therapeutically effective amount of valacyclovir and a delivery agent compound.

The composition of the present invention facilitates the delivery of valacyclovir and increases its bioavailability compared to administration of valacyclovir without the delivery agent compound. The composition is particularly well suited for oral administration. Preferably, the formulations provide acyclovir bioavailability (i.e., AUC) equivalent or substantially equivalent to or better than the current 500 mg valacyclovir formulations marketed as Valtrex® (U.S. FDA NDA Nos. 20-550, 20-550/S21, 20-550/S 9, 20-550/S10, 20-550/S13, 20-550/S16, 20-550/S12, 20-550/S 005) at lower dosing levels of valacyclovir (e.g.) at 300 mg or 350 mg of valacyclovir per dosage unit form).

Also provided is a dosage unit form (erg., an oral dosage unit form) comprising the composition of the present invention and one or more excipients. The dosage unit may be in the form of a liquid or a solid, such as a tablet, capsule or particle, including a powder or sachet.

### BRIEF DESCRIPTION OF THE DRAWINGS

FIGS. 1, 2 and 3 are graphs of the serum concentrations of valacyclovir versus time after oral administration of the pharmaceutical composition of Example 1 to beagles.

FIGS. 4 and 5 are graphs of the serum concentrations of valacyclovir versus time after oral administration of the pharmaceutical composition of Example 2 to beagles.

FIGS. 6 and 7 are graphs of the serum concentrations of valacyclovir versus time after oral administration of the pharmaceutical composition of Example 8 to beagles.

FIGS. 8, 9 and 10 are graphs of the serum concentrations of valacyclovir versus time after oral administration of the pharmaceutical composition of Example 9 to beagles.

FIGS. 11, 12 and 13 are graphs of the serum concentrations of valacyclovir versus time after oral administration of the pharmaceutical composition of Example 10 to beagles.

FIGS. 14 and 15 are graphs of the serum concentrations of valacyclovir versus time after oral administration of the pharmaceutical composition of Example 11 to beagles.

FIG. 16 is a graph of the serum concentrations of valacyclovir versus time after oral administration of the pharmaceutical composition of Example 12 to beagles.

FIGS. 17 and 18 are graphs of the serum concentrations of valacyclovir versus time after oral administration of the pharmaceutical composition of Example 13 to beagles.

### DETAILED DESCRIPTION OF THE INVENTION

### Definitions

The term “hydrate” as used herein includes, but is not limited to, (i) a substance containing water combined in the molecular form and (ii) a crystalline substance containing one or more molecules of water of crystallization or a crystalline material containing free water.

The term “solvate” as used herein includes, but is not limited to, a molecular or ionic complex of molecules or ions of a solvent with molecules or ions of the delivery agent compound, or salt, hydrate or solvate thereof.

The term “delivery agent compound” refers to any of the delivery agent compounds disclosed herein.

The term “SNAC” refers to N-(8-[2-hydroxybenzoyl]-amino)caprylic acid, and pharmaceutically acceptable salts thereof. Unless otherwise noted, the term “SNAC” refers to all amorphous and polymorphic forms of SNAG, such as SNAC trihydrate and those described in U.S. Ser. Nos. 60/619,418 and 60/569,476, both of which are hereby incorporated by reference. The term “SNAC trihydrate” as used herein refers to a crystalline form of SNAC in which three molecules of water are associated with each molecule of SNAC. SNAG can be prepared by the procedures described in U.S. Pat. No. 5,650,386 and International Publication Nos. WO00/46182 and WO00/59863.

The term “SNAD” refers to N-(10-[2-hydroxybenzoyl]-amino)decanoic acid, and pharmaceutically acceptable salts thereof.

Unless otherwise noted, the term “4CNAB” refers to 4-[(4-chloro-2-hydroxy-benzoyl)amino]butanoic acid (also known as 4-[(2-hydroxy-4-chlorobenzoyl)amino]butanoate), and pharmaceutically acceptable salts thereof.

An “effective amount of valacyclovir” is an amount of valacyclovir which is effective to treat or prevent a condition in a living organism to whom it is administered over some period of time, e.g., provides a therapeutic effect during a desired dosing interval.

An “effective amount of delivery agent” is an amount of the delivery agent which enables and/or facilitates the absorption of a desired amount of valacyclovir via any route of administration (such as those discussed in this application including, but not limited to, the oral (e.g., across a biological membrane in the gastrointestinal tract), nasal, pulmonary, dermal, buccal (e.g. sublingual), vaginal, and/or ocular route.

The term “mean”, when preceding a pharmacokinetic value (e.g., mean peak) represents the arithmetic mean value of the pharmacokinetic value unless otherwise specified.

As used herein and in the appended claims, the singular forms “a,” “an,” and “the,” include plural referents unless the context clearly indicates otherwise. Thus, for example, reference to “a molecule” includes one or more of such molecules, “a reagent” includes one or more of such different reagents, reference to “an antibody” includes one or more of such different antibodies, and reference to “the method” includes reference to equivalent steps and methods known to those of ordinary skill in the art that could be modified or substituted for the methods described herein.

The term “about” generally means within 10%, preferably within 5%, and more preferably within 1% of a given value or range.

The terms “alkyl” and “alkenyl” as used herein include linear and branched alkyl and alkenyl substituents, respectively.

The phrase “pharmaceutically acceptable” refers to additives or compositions that are physiologically tolerable and do not typically produce an allergic or similar untoward reaction, such as gastric upset, dizziness and the like, when administered to a mammal.

By “condition or disorder caused by a virus” is meant any condition or disorder in an animal that is caused by, complicated by, or aggravated by a virus. Such conditions or disorders include, but are not limited to, those caused by viruses of the herpes family, for example, herpes simplex 1 and 2 viruses (HSV 1, HSV 2), varicella zoster virus (VZV), cytomegalovirus (CMV), Epstein-Barr virus (EBV), and other herpes virus infections (e.g. feline herpes virus infections).

As used herein, the term “treat” includes one or more of the following.
(a) arresting, delaying the onset (i.e., the period prior to clinical manifestation of a disorder) and/or reducing the risk of developing or worsening a disorder;
(b) relieving or alleviating at least one symptom of a disorder in a mammal, including for example, hypercalcemia; or
(c) relieving or alleviating the intensity and/or duration of a manifestation of a disorder experienced by a mammal including, but not limited to, those which are in response to a given stimulus (e.g., pressure, tissue injury or cold temperature). The term “treat” also includes prophylactically preventing, curing, healing, alleviating, relieving, altering, remedying, ameliorating, improving, or affecting a condition (e.g., a disease), the symptoms of the condition, or the predisposition toward the condition.

The term “iCmax” means the maximum observed plasma concentration, here, the maximum serum concentration of acyclovir expressed in mcg/ml at that point in time when the concentration reaches its highest level in human plasma following a dose of valacyclovir.

The term “AUC” means an area under the concentration curve where the y axis is plasma concentration of acyclovir in mcg/ml and the x axis is time. AUC may be expressed in hr-mcg/ml. For these purposes, area under the curve is from time 0 to the time of the last measurable concentration.

The term “bioavailability” is the percentage of drug which reaches the plasma circulation following administration of a dose of a medication. By definition, the bioavailability for intravenously administered valacyclovir is 100%.

### Valacyclovir and Acyclovir

The term “acyclovir” refers to 9-(2-hydroxyethoxymethyl)guanine. Suitable salts (e.g., pharmaceutically acceptable salts) and esters of acyclovir are described in U.S. Pat. No. 4,199,574, which is hereby incorporated by reference, and include, but are not limited to, sodium acyclovir and acyclovir valerate. Acyclovir also forms acid addition salts, such as with hydrochloric, sulfuric, phosphoric, maleic, fumaric, citric, tartaric, lactic and acetic acid.

A synthesis of acyclovir is disclosed in U.S. Pat. No. 4,199,574, which is hereby incorporated by reference. Acyclovir is commercially available from GIaxoSmithKline (Research Triangle Park, N.C.) under the tradename Zovirax®.

Any prodrug which is converted in vivo to 9-(2-hydroxyethoxymethyl)guanine can also be used. The term “prodrug” as used herein includes pharmaceutically acceptable salts of the drug. Acyclovir prodrugs include, substituted purines of the formula:

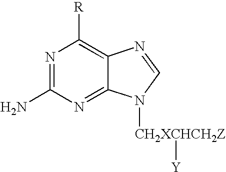

or salts thereof, wherein:
R is hydrogen, hydroxy, or amino;
X is oxygen or sulphur;
Y is hydrogen or hydroxyethyl and
Z is —H, C₁₋₁₆ alkyl, or —OCOCH(R₁)NH₂, wherein R₁ is —CH[CH₃]₂.

Suitable acyclovir prodrugs, include but are not limited to, those described in U.S. Pat. Nos. 4,609,662, 4,758,572 and 4,957,924, all of which are hereby incorporated by reference. A non-limiting example of such a prodrug is 2-[(2-amino-1,6-dihydro-6-oxo-9H-purin-9-yl)methoxy]ethyl ester (valacyclovir) and its pharmaceutically acceptable salts. valacyclovir is commercially available as its hydrochloride salt from GlaxoSmithKline (Research Triangle Park, N.C.) under the tradename Valtrex™.

Therapeutically effective amounts of valacyclovir for use in treatment of all conditions and disorders described herein, is an amount sufficient to suppress or alleviate conditions associated with the viral infection. As will be recognized by those in the field, an effective amount of therapeutic agent will vary with many factors including the potency of the valacyclovir or salt, ester, or prodrug thereof, the age and weight of the patient, and the severity of the condition or disorder to be treated.

The structure of valacyclovir is:

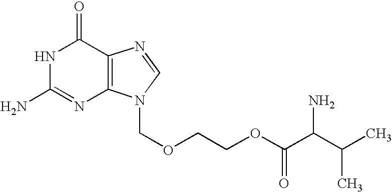

### Delivery Agent Compounds

Preferred delivery agent compounds include, but are not limited to, N-(8-[2-hydroxybenzoyl]amino)caprylic acid, N-(10-[2-hydroxybenzoyl]amino)decanoic acid, 4-[(4-chloro-2-hydroxy-benazoyl)amino]butanioic acid (also known as 4-[(2-hydroxy-4-chlorobenzoyl)amino]butanoate), 8-(N-2-hydroxy-5-chlorobenzoyl)aminocaprylic acid, 8-(N-2-hydroxy-4-methoxybenzoyl)-amino-caprylic acid, and salts (including pharmaceutically acceptable salts) (e.g., sodium or disodium) thereof, and solvates and hydrates thereof. The salt can be, for example, a sodium salt, such as a monosodium or disodium salt.

In one embodiment, the delivery agent compound is 4CNAB and the active agent is valacyclovir HCl. More preferably, the delivery agent compound is the monosodium salt of 4CNAB.

In another preferred embodiment, the delivery agent compound is SNAC and the active agent is valacyclovir HCl. More preferably, the delivery agent compound is the monosodium salt of SNAC.

In another preferred embodiment, the delivery agent is SNAD and the active agent is valacyclovir HCl. More preferably, the delivery agent compound is the disodium salt of SNAD.

In one embodiment of the present invention, the delivery agent compound has the following structure, or a pharmaceutically acceptable salt thereof:

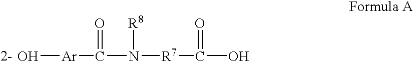

wherein

Ar is phenyl or naphthyl;

Ar is optionally substituted with one or more of —OH, halogen, C₁-C₄ alkyl, C₁-C₄ alkenyl, C₁-C₄ alkoxy or C₁-C₄ haloalkoxy;

R⁷ is C₄-C₂₀(alkyl, C₄-C₂₀ alkenyl, phenyl, naphthyl(C₁-C₁₀ alkyl)phenyl, (C₁-C₁₀ alkenyl)phenyl, (C₁-C₁₀ alkyl)naphthyl, (C₁-C₁₀ alkenyl)naphthyl, phenyl(C₁-C₁₀ alkyl), phenyl(C₁-C₁₀ alkenyl), naphthyl(C₁-C₁₀ alkyl), or naphthyl(C₁-C₁₀ alkenyl);

R⁸ is hydrogen, C₁ to C₄ alkyl, C₂ to C₄ alkenyl, C₁ to C₄ alkoxy, or C₁-C₄ haloalkoxy;

R⁷ is optionally substituted with C₁ to C₄ alkyl, C₂ to C₄ alkenyl, C₁ to C₄ alkoxy, C₁-C₄ haloalkoxy, —OH, —SH, —CO₂R⁹, or any combination thereof;

R⁹ is hydrogen, C₁ to C₄ alkyl, or C₂ to C₄ alkenyl; and

R⁷ is optionally interrupted by oxygen, nitrogen, sulfur or any combination thereof.

In one embodiment, the delivery agent compounds are not substituted with an amino group in the position alpha to the acid group.

According to one embodiment) R⁷ in Formula A is selected from C₈-C₂₀ alkyl, C₉-C₂₀ alkenyl, phenyl, naphthyl, (C₁-C₁₀ alkyl)phenyl, (C₁-C₁₀ alkenyl)phenyl, (C₁-C₁₀ alkyl) naphthyl, (C₁-C₁₀ alkenyl) naphthyl, phenyl(C₁-C₁₀ alkyl), phenyl(C₁-C₁₀ alkenyl), naphthyl(C₁-C₁₀ alkyl), and naphthyl(C₁-C₁₀ alkenyl).

According to another embodiment, R⁷ in Formula A is selected from C₈-C₂₀ alkyl, and C₈-C₂₀ alkenyl.

In another embodiment of the present invention, the delivery agent compound has the following structure, or a pharmaceutically acceptable salt thereof:

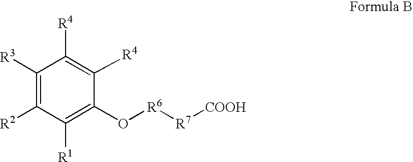

wherein

R¹, R², R³, and R⁴ are independently H, —OH, halogen, C₁-C₄ alkyl, C₂-C₄ alkenyl, C₁-C₄ alkoxy, —C(O)R⁸, —NO₂, —NR⁹R¹⁰, or —N⁺R⁹R¹⁰R¹¹ (R¹²);

R⁵ is H, —OH, —NO₂, halogen, —CF₃, —NR¹⁴R¹⁵, —N⁺R¹⁴R¹⁵, R¹⁶ (R¹³)⁻, amide, C₁-C₁₂ alkoxy, C₁-C₁₂ alkyl, C₂-C₁₂ alkenyl, carbamate, carbonate, urea, or —C(O)R¹⁸;

R⁵ is optionally substituted with halogen, —OH, —SH, or —COOH;

R⁵ is optionally interrupted by O, N, S, or —C(O)—;

R⁶ is a C₁-C₁₂ alkylene, C₂-C₁₂ alkenylene, or arylene;

R⁶ is optionally substituted with a C₁-C₄ alkyl, C₂-C₄ alkenyl), C₁-C₄ alkoxy, —OH, —SH, halogen, —NH₂, or —CO₂R⁸;

R⁶ is optionally interrupted by O or N;

R⁷ is a bond or arylene;

R⁷ optionally substituted with —OH, halogen, —C(O)CH₃, —NR¹⁰R¹¹, or —N⁺R¹⁰R¹¹R¹²(R¹³);

each occurrence of R⁸ is independently H, C₁-C₄ alkyl, C₂-C₄ alkenyl, or —NH₂;

R⁹, R¹⁰, R¹¹ and R¹² independently H or C₁-C₁₀ alkyl;

R¹³ is a halide, hydroxide, sulfate, tetrafluoroborate, or phosphate;

R¹⁴, R¹⁵ and R¹⁶ are independently H, C₁-C₁₀ alkyl, C₁-C₁₀ alkyl substituted with —COON, C₂-C₁₂ alkenyl, C₂-C₁₂ alkenyl substituted with COOH, or —C(O)R¹⁷;

R¹⁷ is —OH, C₁-C₁₀ alkyl, or C₂-C₁₂ alkenyl; and

R¹⁸ is H, C₁-C₆ alkyl, —OH, —NR¹⁴R¹⁵, or N⁺R¹⁴R¹⁵R¹⁶(R¹³)⁻.

In one particular embodiment, when R¹, R², R³, R⁴, and R⁵ are H, and R⁷ is a bond then R⁶ is not a C₁-C₆, C₉ or C₁₀ alkyl.

In another embodiment, when R¹, R², R³, and R⁴ are H, R⁵ is —OH, and R⁷ is a bond then R⁶ is not a C₁-C₃ alkyl.

In yet another embodiment, when at least one of R¹, R², R³, and R⁴ is not H, R⁵ is —OH, and R⁷ is a bond, then R⁶ is not a C₁-C₄ alkyl.

In yet another embodiment, when R¹, R², and R³ are H, R⁴ is —OCH₃, R⁵ is —C(O)CH₃, and R⁶ is a bond then R⁷ is not a C₃ alkyl.

In yet another embodiment, when R¹, R², R⁴, and R⁵ are H, R³ is —OH, and R⁷ is a bond then R⁶ is not a methyl.

In yet another embodiment, R⁶ of Formula B is a C₈-C₁₂ alkylene, C₈-C₁₂ alkenylene, or arylene.

In yet another embodiment of the present invention, the delivery agent compound has the following structure or a pharmaceutically acceptable salt thereof:

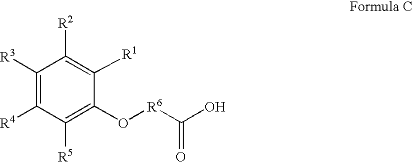

wherein

R¹, R², R³, R⁴ and R⁵ are independently H, —CN, —OH, —OCH₃, or halogen, at least one of R¹, R², R³, R⁴ and R⁵ being —CN; and

R⁶ is a C₁-C₁₂ linear or branched alkylene, a C₁-C₁₂ linear or branched alkenylene, a C₁-C₁₂ linear or branched arylene, an alkyl(arylene) or an aryl(alkylene).

According to one embodiment, when R¹ is —CN, R⁴ is 11 or —CN, and R², R³, and R⁵ are H, then R⁶ is not methylene ((CH₂)₁).

In another embodiment, R⁶ of Formula C is a C₈-C₁₂ linear or branched alkylene, a C₈-C₁₂ linear or branched alkenylene, an arylene, an alkyl(arylene) or an aryl(alkylene).

In yet another embodiment, R⁶ of Formula C is a C₈-C₁₂ linear or branched alkylene, a C₈-C₁₂ linear or branched alkenyl one

Other suitable delivery agent compounds are disclosed in U.S. Pat. No. 6,627,228, which is hereby incorporated by reference.

In embodiments of the present invention, delivery agent compounds to be used in the topical composition along with the acyclovir compound include, but are not limited to, a polymeric delivery agent comprising a polymer conjugated to a modified amino acid or derivative thereof via a linkage group selected from the group consisting of —NHC(O)NH—, —C(O)NH—, —NHC(O)—, —OOC—, —COO—, —NHC(O)O—, —OC(O)NH—, —CH₂NH—, —NHCH₂—, —CH₂NHC(O)O—, —OC(O)NHCH₂—, —CH₂NHCOCH₂O—, —OCH₂C(O)NHCH₂—, —NHC(O)CH₂O—, —OCH₂C(O)NH—, —NH—, —O—, and carbon-carbon bond. In one embodiment, the polymeric delivery agent is not a polypeptide or polyamino acid. In another embodiment, the modified amino acid has the structure of formula A, B, or C. In one embodiment, the polymeric delivery agent includes a modified amino acid having the structure:

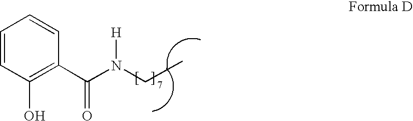

which is conjugated via a —COO group to a polymer having monomers derived from polyethylene glycol.

In one embodiment, the polymeric delivery agent is a modified amino acid having the structure of Formula D conjugated via a —COO group to a polymer having the structure:

—CH₂CH₂—O(CH₂CH₂O)ₓCH₂CH₂O—Y,

wherein

x is from 1-14; and

Y is H or CH₃.

According to one embodiment, the polymeric delivery agent is compound having the structure of Formula D conjugated via a —COO group to a polymer having the structure:

—CH₂CH₂O(CH₂CH₂O)ₓCH₂CH₂O—Y,

wherein
x is 1-9; and
Y is CH₃ or H. For example, the polymeric delivery agent can be 8-(2-hydroxybenzoylamino)-octanoic acid 2-{2-[2-(2-{2-[2-(2-methoxyethoxy)ethoxy]ethoxy}-ethoxy)ethoxy]ethoxy}ethyl ester.

In one embodiment, the delivery agent compound is PEGylated SNAC with an average of about 6-9 or about 7-8 (e.g. 7.3) repeating ethylene oxide groups and having a molecular weight of about 500-800 (e.g. 600) daltons.

Delivery agent compounds of the present invention include compounds as shown below and pharmaceutically acceptable salts thereof:

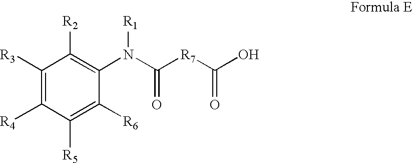

wherein:

R₁ is —(C₂)ₘ—R₈, wherein m=0 or 1;

R₂-R₆ are independently selected from hydrogen, hydroxyl, halogen, C₁-C₄ alkyl, C₂-C₄ alkenyl, C₂-C₄ alkynyl, C₁-C₄ alkoxy, and cyano;

R₇ is selected from C₁-C₁₀ alkyl, C₂-C₁₀ alkenyl, and C₂-C₁₀ alkynyl;

R₈ is selected from cyclopentyl, cyclohexyl and phenyl, wherein when R₈ is a phenyl, m=1; and

R₈ is optionally substituted with C₁-C₄ alkyl, C₁-C₄ alkoxy, halogen or hydroxyl, or a combination thereof.

Other delivery agent compounds of the present invention include those of the formula:

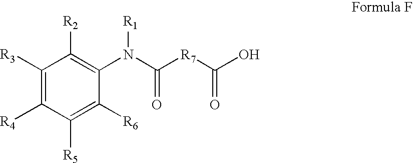

and pharmaceutically acceptable salts thereof, wherein:

R₁ is a C₁-C₆ alkyl, or C₂-C₆ alkenyl,

R₂-R₆ are independently chosen from the group consisting of hydrogen, hydroxyl, halogen, C₁-C₄ alkyl, C₂-C₄ alkenyl, C₂-C₄ alkynyl, C₁-C₄ alkoxy, and cyano, and

R₇ is selected from the group consisting of C₁-C₁₀ alkyl, C₂-C₁₀ alkenyl, and C₂-C₁₀ alkynyl.

Other delivery agent compounds of the present invention include those of the formula:

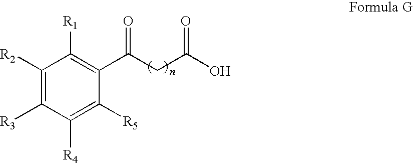

and pharmaceutically acceptable salts thereof, wherein

n 1 to 9, and

R₁ to R₅ are independently hydrogen, C₁ to C₄ alkyl, C₁ to C₄ alkoxy, C₂ to C₄ alkenyl, halogen, hydroxyl, —NH—C(O)—CH₃, or O—C₆H₅.

Other delivery agent compounds of the present invention include those of the formula:

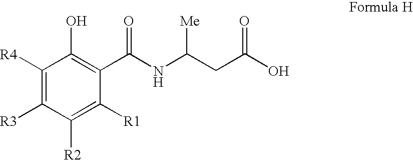

and pharmaceutically acceptable salts thereof, wherein

R₁ to R₄ are independently hydrogen, C₁ to C₄ alkyl, C₂ to C₄ alkenyl, halogen, C₁ to C₄ alkoxy, or hydroxyl.

Other delivery agent compounds of the present invention include those of the formula:

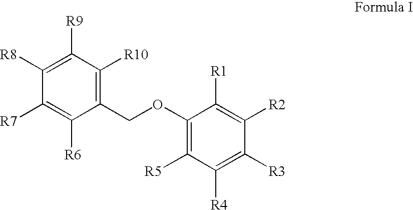

and pharmaceutically acceptable salts thereof, wherein

one of R₁ to R₅ has the generic structure

—(CH₂)n-COOH

where n=0−6;

the remaining four members of R₁ to R₅ are independently hydrogen, C₁ to C₄ alkyl, C₂ to C₄ alkenyl, halogen, C₆ to C₄ alkoxy, or hydroxyl; and

R₆-R₁₀ are independently hydrogen, C₁ to 64 alkyl, C₂ to C₄ alkenyl, halogen, C₁ to C₄ alkoxy, or hydroxyl.

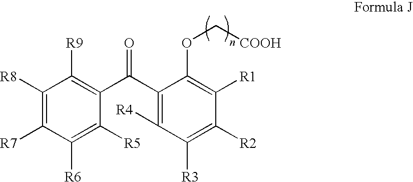

and pharmaceutically acceptable salts thereof, wherein

n=1 to 9; and

R₁ to R₉ are independently hydrogen, C₁ to C₄ alkyl, C₂ to C₄ alkenyl, halogen, C₁ to C₄ alkoxy, or hydroxyl.

Other delivery agent compounds of the present invention include those of the formula:

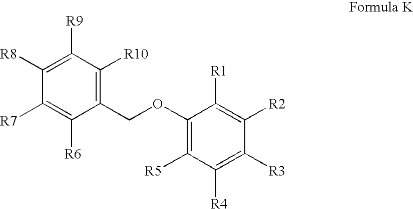

and pharmaceutically acceptable salts thereof, wherein

R₁-R₅ are independently hydrogen, C₁ to C₄ alkyl, C₂ to C₄ alkenyl, halogen, C₁ to C₄ alkoxy, hydroxyl, or —O—(CH₂)n-COOH (where n is 1 to 12);

at least one of R₁ to R₅ has the generic structure

—O—(CH₂)n—COOH

where n=1−12; and

R₆-R₁₀ are independently hydrogen, C₁ to C₄ alkyl, C₂ to C₄ alkenyl, halogen, C₁ to C₄ alkoxy, or hydroxyl. International Application Nos. PCT/US2005/017339 and PCT/US2005/017309, filed May 16, 2005 (Attorney Docket Nos. 01946/2201284-WO0 and 01946/2201285-WO0) and their priority documents, U.S. Provisional Application Nos. 60/576,088, filed Jun. 1, 2004, U.S. Provisional Application No. 60/576,397, filed Jun. 1, 2004, U.S. Provisional Application No. 60/576,105, filed Jun. 1, 2004, U.S. Provisional Application No. 60/571,090, filed May 14, 2004, U.S. Provisional Application No. 60/571,092, filed May 14, 2004, U.S. Provisional Application No. 60/571,195, filed May 14, 2004, U.S. Provisional Application No. 60/571,194, filed May 14, 2004, U.S. Provisional Application No. 60/571,093, filed May 14, 2004, U.S. Provisional Application No. 60/571,055, filed May 14, 2004, U.S. Provisional Application No. 60/571,151, filed May 14, 2004, U.S. Provisional Application No. 60/571,315, filed May 14, 2004, U.S. Provisional Application No. 60/571,144, filed May 14, 2004, and U.S. Provisional Application 60/571,089, filed May 14, 2004, are hereby incorporated by reference in their entirety.

The delivery agent compound may also be any of those described in U.S. Pat. Nos. 6,699,467, 6,663,898, 6,693,208, 6,693,073, 6,693,898, 6,663,887, 6,646,162, 6,642,411, 6,627,228, 6,623,731, 6,610,329, 6,558,706, 6,525,020, 6,461,643, 6,461,545, 6,440,929, 6,428,780, 6,413,550, 6,399,798, 6,395,774, 6,391,303, 6,384,278, 6,375,983, 6,358,504, 6,346,242, 6,344,213, 6,331,318, 6,313,088, 6,245,359, 6,242,495, 6,221,367, 6,180,140, 6,100,298, 6,100,285, 6,099,856, 6,090,958, 6,084,112, 6,071,510, 6,060,513, 6,051,561, 6,051,258, 6,001,347, 5,990,166, 5,989,539, 5,976,569, 5,972,387, 5,965,121, 5,962,710, 5,958,451, 5,955,503, 5,939,381, 5,935,601, 5,879,681, 5,876,710, 5,866,536, 5,863,944, 5,840,340, 5,824,345, 5,820,881, 5,811,127, 5,804,688, 5,792,451, 5,776,888, 5,773,647, 5,766,633, 5,750,147, 5,714,167, 5,709,861, 5,693,338, 5,667,806, 5,650,386, 5,643,957, 5,629,020, 5,601,846, 5,578,323, 5,541,155, 5,540,939, 5,451,410, 5,447,728, 5,443,841, and 5,401,516; International Publication Nos. WO94/23767, WO95/11690, WO95/28920, WO95/28838, WO96/10396, WO6/09813, WO96/12473, WO97/36480, WO2004/4104018, WO2004080401, WO2004062587, WO2003/057650, WO2003/057170, WO2003/045331, WO 2003/045306, WO2003/026582, WO2002/100338, WO2002/070438, WO2002/069937, WO 02/20466, WO 02/19969, WO02/16309, WO02/15959, WO02/02509, WO 01/92206, WO 01/70219, WO 01/51454, WO 01/44199, WO 01/34114, WO 01/32596, WO 01/32130, WO 00/07979, WO 00/06534, WO 00/06184, WO 00/59863, WO 00/59480, WO 00/50386, WO 00/48589, WO 00/47188, WO 000/46182, WO 00/40203, WO99/16427, WO 98/50341, WO 98/49135, WO 98/34632, WO 98/25589, WO 98/21951, WO 97/47288, WO97/31938, WO 97/10197, WO 96/40076, WO 96/40070, WO 96/39835, WO 96/33699, WO 096/30036, WO 96/21464, WO96/12475, and WO 96/12474; and U.S. Published Application Nos. 20040110839, 20040106825, 20040068013, 20040062773, 20040022856, 20030235612, 20030232085, 20030225300, 20030198658, 20030133953, 20030078302, 20030072740, 20030045579, 20030012817, 20030008900, 20020155993, 20020127202, 20020120009, 20020119910, 20020102286, 20020065255, 20020052422, 20020040061, 20020028250, 20020013497, 20020001591, 20010039258, and 20010003001. Each of the above listed U.S. patents and U.S. and International published applications are herein incorporated by reference.

Non-limiting examples of delivery agent compounds include, but are not limited to, N-(8-[2-hydroxybenzoyl]-amino)caprylic acid, N-(10-[2-hydroxybenzoyl]-amino)decanoic acid, 8-(2-hydroxy-4-methoxybenzoylamino)octanoic acid, 8-(2,6-dihydroxybenzoylamino)octanoic acid, 8-(2-hydroxy-5-bromobenzoylamino)octanoic acid, 8-(2-hydroxy-5-chlorobenzoylamino)octanoic acid, 8-(2-hydroxy-5-iodobenzoylamino)octanoic acid, 8-(2-hydroxy-5-methylbenzoylamino)octanoic acid, 8-(2-hydroxy-5-fluorobenzoylamino)octanoic acid, 8-(2-hydroxy-5-methoxybenzoylamino)octanoic acid, 8-(3-hydroxyphenoxy)octanoic acid, 8-(4-hydroxyphenoxy)octanoic acid, 6-(2-cyanophenoxy)hexanoic acid, S-(2-Hydroxyphenoxy)octyl-diethanolamine, 8-(4-hydroxyphenoxy)octanoate, 8-(4-hydroxyphenoxy)octanoate, 8-(2-hydroxy-4-methoxybenzoylamino)octanoic acid, 8-(2-hydroxy-5-methoxybenzoylamino)-octanoic acid, and salts thereof. Preferred salts include the monosodium and disodium salts.

The delivery agent compounds may be in the form of the carboxylic acid or pharmaceutically acceptable salts thereof, such as sodium salts, and hydrates and solvates thereof. The salts may be mono- or multi-valent salts, such as monosodium salts and disodium salts. The delivery agent compounds may contain different counter ions chosen for example due to their effect on modifying the dissolution profile of the delivery agent compound.

The delivery agent compounds may be prepared by methods known in the art, such as those discussed is) the aforementioned publications (e.g., International Publication Nos. WO 98/34632, WO 00/07979, WO 01/44199, WO 01/32596, WO 02/20466, WO 03/045306) and U.S. Published Application Nos. 20050272639, 20050250852, 20040048777. SNAC, SNAD, 4CNAD and pharmaceutically acceptable salts thereof may be prepared by methods known in the alt, such as those described in U.S. Pat. Nos. 5,650,386 and 5,866,536.

Salts of the delivery agent compounds of the present invention may be prepared by methods known in the art. For example, sodium salts may be prepared by dissolving the delivery agent compound in ethanol and adding aqueous sodium hydroxide. Disodium salts may be prepared as disclosed in U.S. Published Application No. 20040106825, which is hereby incorporated by reference in its entirety.

The delivery agent compound may be purified by recrystallization or by fractionation on one or more solid chromatographic supports, alone or linked in tandem. Suitable recrystallization solvent systems include, but are not limited to, acetonitrile, methanol, and tetrahydrofuran. Fractionation may be performed on a suitable chromatographic support such as alumina, using methanol/n-propanol mixtures as the mobile phase; reverse phase chromatography using trifluoroacetic acid/acetonitrile mixtures as the mobile phase; and ion exchange chromatography using water or an appropriate buffer as the mobile phase. When anion exchange chromatography is performed, preferably a 0-500 mm sodium chloride gradient is employed.

### Delivery Systems

The composition of the present invention comprises one or more delivery agent compounds of the present invention and valacyclovir. The delivery agent compound and valacyclovir acyclovir are typically mixed prior to administration to form an administration composition.

One embodiment of the present invention provides a pharmaceutical formulation (e.g., an oral pharmaceutical formulation) comprising valacyclovir and one or more of the delivery agents chosen from the group consisting of SNAC, SNAD, and 4CNAB. In one embodiment, the delivery agent compound is 4CNAB (e.g. the sodium salt of 4CNAB).

Yet another embodiment is a pharmaceutical formulation comprising from about 100 mg to about 2000 mg of valacyclovir and from about 25 mg to about 500 mg of SNAC.

Yet another embodiment is a pharmaceutical formulation comprising from about 300 mg to about 350 mg (e.g., 300 mg) of valacyclovir and from about 300 mg to about 350 mg of SNAC (e.g., the monosodium salt of SNAC).

Yet another embodiment is a pharmaceutical formulation comprising from about 100 mg to about 2000 mg of valacyclovir and from about 25 mg to about 500 mg of SNAD.

Yet another embodiment is a pharmaceutical formulation comprising from about 300 mg to about 350 mg of valacyclovir and from about 300 mg to about 350 mg of SNAP (e.g., the disodium salt of SNAD).

Yet another embodiment is a pharmaceutical formulation comprising from about 100 mg to about 200 mg of valacyclovir and from about 25 mg to about 500 mg of 4CNAB.

Yet another embodiment is a pharmaceutical formulation comprising about 300 mg to about 350 mg of valacyclovir and from about 300 mg to about 350 mg of 4CNAB (e.g., the monosodium salt of 4CNAB).

Yet another embodiment is a formulation comprising valacyclovir and a delivery agent compound selected form the group consisting of SNAC, SNAD and 4CNAB and valacyclovir in which the mean AUC is from about 4 to about 7 hr*mcg/ml following a single dose of valacyclovir when administered to a human.

Yet another embodiment is a formulation comprising valacyclovir and a delivery agent compound selected form the group consisting of SNAC, SNAD and 4CNAB and valacyclovir in which the mean time to Cmax (Tₘₐₓ) is at from about 50 minutes to about 60 minutes following a single oral dose to a human.

Yet another embodiment is a composition comprising valacyclovir and SNAG (e.g. the monosodium salt of SNAC) in a weight ratio of about 1:5, or 0.25:80, or 1:1.

According to one embodiment, the valacyclovir (or a salt, ester, prodrug thereof) is administered (e.g. peripherally) at a dose of about 500 mg to about 2000 mg per day (based on the weight of acyclovir). The valacyclovir and delivery agent compound may be administered separately or together with one or more other active agents. For example, the valacyclovir and delivery agent compound may be administered separately or together with compounds or compositions that exhibit antiviral activity, such as compounds used to treat retroviral infections (particularly HIV infections), e.g., 3′-azido-3′-deoxythymidine (AZT) and/or compounds or compositions that exhibit activity as ribonucleotide reductase inhibitors. Suitable ribonucleotide reductase inhibitors include, but are not limited to, thiocarbonohydrazone ribonucleotide reductase inhibitors, such as those disclosed in U.S. Pat. No. 5,393,883, which is hereby incorporated by reference.

Yet another embodiment is a composition comprising valacyclovir and SNAD (e.g. the disodium salt of SNAD) in a ratio of about 1:5, or 0.25:80, or 1:1.

Yet another embodiment is a composition comprising valacyclovir and 4CNAB (e.g. the monosodium salt of 4CNAB) in a ratio of about 1:5, or 0.25:80, or 1:1.

The administration compositions may be in the form of a liquid. The solution medium may be water, 25% aqueous propylene glycol, or phosphate buffer. Other dosing vehicles include polyethylene glycol. Dosing solutions may be prepared by mixing a solution of the delivery agent compound with a solution of the active agent, just prior to administration. Alternately, a solution of the delivery agent compound (or valacyclovir) may be mixed with the solid form of valacyclovir (or delivery agent compound). The delivery agent compound and valacyclovir may also be mixed as dry powders. The delivery agent compound and valacyclovir can also be admixed during the manufacturing process.

The dosing solutions may optionally contain additives such as phosphate buffer salts, citric acid, glycols, or other dispersing agents. Stabilizing additives may be incorporated into the solution, preferably at a concentration ranging between about 0.1 and 20% (w/v).

For example the composition is useful in the invention can be provided as parenteral compositions (e.g., injection or infusion). According to one embodiment, the composition is suspended in an aqueous delivery agent compound, such as in an isotonic buffer solution at a pH of about 3.0 to about 8.0. Suitable buffers include, but are not limited to, sodium citrate citric acid, sodium phosphate phosphoric acid, and sodium acetate/acetic acid buffers.

A form of repository or “depot” slow release preparation may also be used so that therapeutically effective amounts of the preparation are delivered into the bloodstream over many hours or days following transdermal injection or delivery. Furthermore, gastric retention delivery systems may be employed, such as disclosed in International Publication No. WO 2006/084164, which is hereby incorporated by reference.

The administration compositions may alternately be in the form of a solid, such as a tablet, capsule or particle, such as a powder or sachet. Solid dosage forms may be prepared by mixing the solid form of the compound with the solid form of valacyclovir. Alternately, a solid may be obtained from a solution of compound and valacyclovir by methods known in the art, such as freeze-drying (lyophilization), precipitation, crystallization and solid dispersion. Alternatively, the administration can be a semi-solid, in the form of a gel, paste, colloid, gelatin, emulsion, suspension and the like.

### Excipients

Embodiments of the present invention provide oral pharmaceutical compositions comprising (a) at least one delivery agent compound, (b) valacyclovir or a salt ester, or prodrug thereof, and (c) at least one excipient. In various embodiments of the present invention, the excipient may provide increased acyclovir plasma concentrations, as compared to the same pharmaceutical formulations without the excipient.

Examples of excipients that may be used in pharmaceutical compositions of the present invention include, but are not limited to, enzyme inhibitors, binders, coatings, disintegrants, lubricants, antiadherents, glidents, emulsifiers, fillers, diluents, sorbents, sweetners, flavorants and preservatives. Standard pharmaceutical formulation techniques may be used, such as those disclosed in Remington's Pharmaceutical Sciences, Mack Publishing Company, Easton, Pa. (1990), which is hereby incorporated by reference.

### Enzymes Inhibitors

The administration compositions of the present invention may also include one or more enzyme inhibitors. Such enzyme inhibitors include, but are not limited to, compounds such as actinonin or epiactinonin and derivatives thereof. Other enzyme inhibitors include, but are not limited to, aprotinin (Trasylol) and Bowmani-Birk inhibitor.

### Binders

Binders may be employed, for example to impart cohesive qualities and ensure mechanical strength of the pharmaceutical composition. Examples of binders include sugars, glucose, sucrose, lactose, or sugar alcohols like xylitol, sorbitol or maltitol, polyethylene glycol, polyvinylpyrrolidone (Povidone, such as K30 or K90; a variety of Povidones are known commercially under the tradename Kollidon®), acacia gum, natural and synthetic gums, gelatin, starches such as corn starch and potato starch, pregelatinized starch, sodium alginate, magnesium aluminium silicate, tragacanth, cellulose or modified cellulose such as hydroxypropyl cellulose, microcrystalline cellulose (Ceolus®), methylcellulose, sodium carboxylethylcellulose, hydroxypropyl methylcellulose and ethylcellulose.

In an alternative embodiment, pharmaceutical compositions of the present invention include solution or dry binders. Examples of solution binders, for example, for use in wet granulation processes by way of example include, but are not limited to, gelatin, cellulose, cellulose derivatives, polyvinylpyrrolidone (Povidone, such as K30 or K90; a variety of Povidones are also known commercially under the tradename Kollidon®), starch, sucrose or polyethylene glycol as examples. Dry binders, added to a powder blend either after a wet granulation step, or as part of a direct powder compression (DC) formulation may include cellulose, methyl cellulose, polyvinylpyrrolidone (Povidone, such as K30 or K90) or polyethylene glycol such as polysorbate 80 (Tween® 80) as examples. Other types of binders/compression agents include dibasic calcium phosphate, dihydrate (Encompress®) or anhydrous dibasic calcium phosphate (Anhydrous Encompress®)

### Coatings

Tablet coatings protect tablet ingredients fi-o deterioration by moisture in the air and facilitate oral dosing and patient compliance. In one embodiment of the present invention, pharmaceutical compositions include a cellulose (plant fiber) film coating, which is free of sugar and potential allergens. Alternatively, other coating materials may be used, for example synthetic polymers, corn protein zein or other polysaccharides.

### Disintegrants

While not being bound by any particular theory, disintegrants are believed to aid in oral drug delivery by facilitating the breaking-up or disintegration of the tablet or granulated material, thereby assisting in the dissolution process. Examples of disintegrants include, but are not limited to, starch, sodium starch glycolate, sodium carboxymethyl starch, cellulose, sodium carboxylmethyl cellulose, calcium carboxymethyl cellulose, agar, alginic acid and the sodium salt thereof, croscarmellose sodium, crospovidone, polyvinylpyrrolidone (Povidone, such as K30 or K90; a variety of Povidones are also known commercially under the tradename Kollidon®), crosslinked polyvinyl pyrrolidone, methyl cellulose, microcrystalline cellulose (Ceolus®), lower alkyl-substituted hydroxypropyl cellulose, starch, pregelatinized starch, effervescent mixtures, clays, and ion exchange resins.

### Lubricants, Antiadherents, Glidants

Lubricants, antiadherents and glidants include magnesium and calcium stearate, other metallic stearates, sodium stearyl fumarate, fats such as vegetable stearin, stearic acid, silicone fluid, talc, waxes, oils, fumed silicon dioxide (such as Aerosil® 200) and colloidal silica. Also liquid lubricants such as polyethylene glycol such as polysorbate 80 (Tween® 80) and vegetable oils such as peanut oil, cottonseed oil, sesame oil, olive oil, corn oil and oil of theobroma may be used in pharmaceutical compositions of the present invention. Fumed silicon dioxide (SiO₂) may be used in the pharmaceutical compositions of the present invention to aid tablet processing. One commercial example of this product is known as Aerosil® 200. Talc may also be used in compositions of the present invention as a glidant.

### Emulsifiers

The emulsifiers or suspending agents can be used as vehicles for dispersion and may be included pharmaceutical compositions of the present invention. Emulsifiers useful in embodiments of the present invention include, but are not limited to, monoglyceride compounds, diglyceride compounds, triglycecride compounds, glycerol, polyethylene glycols such as polysorbate 80 (Tween® 80), phospholipids, gum acacia, agar, petrolatum, lanolin, dimethyl sulfoxide (DMSO), normal saline (NS), phosphate buffered saline (PBS), sodium alginate, bentonite, carbomer, carboxymethyl-cellulose, carrageenan, powdered cellulose, cholesterol, gelatin, hydroxyethyl cellulose, hydroxypropyl cellulose, hydroxypropyl methylcellulose, methylcellulose, octoxynol 9, oleyl alcohol, polyvinyl alcohol, polyvinylpyrrolidone (Povidone, such as K30 or K90; a variety of Povidones are also known commercially under the tradename Kollidon®), propylene glycol monostearate, sodium lauryl sulfate, sorbitan esters, stearyl alcohol, tragacanth, xanthan gum, chondrus, glycerin, trolamine, avacado oil, coconut oil, coconut butter, propylene glycol, ethyl alcohol, malt and malt extract.

### Fillers/Diluents

Fillers and diluents fill out the size of a tablet or capsule, increasing the bulk volume and facilitating a final product that has the proper volume for patient handling. An acceptable filler will be inert, compatible with the other components of the formulation, non-hygroscopic, soluble, compactable and preferably tasteless or pleasant tasting, Acceptable fillers, include, but are not limited to, plant cellulose, dibasic calcium phosphate, vegetable fats and oils, (e.g., soft gelatin capsules containing vegetable oil). Other examples of fillers include: lactose (monohydrate, spray-dried monohydrate, anhydrous and the like), mannitol, xylitol, dextrose, sucrose, glucose, sorbitol, microcrystalline cellulose (Ceolus®), starch, calcium carbonate and dibasic calcium phosphate dihydrate.

The formulation may also contain powders such as chalk, talc, fullers earth, kaolin, starch, gums, colloidal silicon dioxide, sodium polyacrylate, tetra alkyl ammonium smectites, trialkyl aryl ammonium smectites, clinically modified magnesium aluminum silicate, organically modified montmorillonite clay, hydrated aluminum silicate, fumed silica, carboxyvinyl polymer, sodium carboxymethyl cellulose, ethylene glycol monostearate, and combinations thereof.

### Sorbents

Examples of sorbents that may be used in pharmaceutical compositions of the present invention include, but are not limited to, charcoal.

### Sweeteners/Flavorants

Sweetners or flavorants such as aspartame, sucrose or mint flavoring may be added.

### Preservatives

Some typical preservatives contemplated by the pharmaceutical formulations in the present invention include antioxidants such vitamin A, vitamin E, vitamin C, and selenium, the amino acids cysteine and methionine, citric acid, sodium citrate or synthetic preservatives like methyl paraben and propyl paraben.

### Dosage Amounts

The amount of valacyclovir used in an administration composition of the present invention is an amount effective to treat the target indication. However, the amount can be less than that amount when the composition is used in a dosage unit form because the dosage unit form may contain a divided effective amount (e.g., the dosage unit form may contain ½ the effective amount, and two dosage unit forms are administered at one time). The total effective amount can then be administered in cumulative units containing, in total, an effective amount of valacyclovir. Moreover, those skilled in the filed will recognize that an effective amount of acyclovir will vary with many factors including the age and weight of the patient, the patient's physical condition, especially renal function, as well as other factors.

The total amount of valacyclovir to be used of can be determined by methods known to those skilled in the art. However, because the compositions of the invention may deliver valacyclovir more efficiently than compositions containing valacyclovir without the delivery agent compound, lower amounts of valacyclovir than those used in prior dosage unit forms or delivery systems can be administered to the subject, while still achieving the same blood levels and/or therapeutic effects.

The desired dose may be administered either as a single or divided dose.

Generally an effective amount of delivery agent to facilitate the delivery of valacyclovir is administered with valacyclovir. According to one embodiment, the amount of delivery agent to valacyclovir on a molar basis ranges from about 20:1 to about 1:1 or 0.25:1, or from about 10:1 to about 2:1, or from about 5:1 to about 2:1.

Dosage unit forms can also include any one or combination of excipients, diluents, disintegrants, lubricants, plasticizers, colorants, flavorants, taste-masking agents, sugars, sweeteners, salts, and dosing vehicles, including, but not limited to, water, 1,2-propane diol, ethanol, olive oil, or any combination thereof.

The compositions of the subject invention are useful for administering biologically or chemically active agents to any animals, including but not limited to birds such as chickens; fish, reptiles, mammals, such as rodents, cows, pigs, dogs, cats, primates, and particularly humans, and insects.

### Methods of Treatment

The composition of the present invention can treat any disorder which is treatable with valacyclovir or its salts (e.g., acyclovir sodium) or prodrugs (e.g., valacyclovir), including those described in the Physicians' Desk Reference (58^{th} Ed., 2004, Medical Economics Company, Inc., Montvale, N.J.). Such disorders include, but are not limited to, those described above or in the patents or other publications above. Non-limiting examples are:
(1) herpes simplex 1 virus (HSV 1),
(2) herpes simplex 2 virus (HSV 2),
(3) varicella zoster virus (VZV),
(4) cytomegalovirus (CMV),
(5) Epstein-Barr virus (EBV),
(6) other herpes virus infections (e.g. feline herpes virus infections),
(7) herpetic karatitis,
(8) herpetic encaphalitis,
(9) cold sores and genital infections (caused by herpes simplex),
(10) chicken pox,
(11) shingles (caused by varicella zoster),
(12) CMV-pneumonia and retinitis, particularly in immunocompromised patients including renal and bone marrow transplant patients and patients with Acquired Immune Deficiency Syndrome (AIDS),
(13) Epstein-Barr virus (EVB) caused infectious mononucleosis, nasopharyngeal cancer, immunoblastic lymphoma, Burkitt's lymphoma and hairy leukoplakia,
(14) herpes zoster, and
(15) initial episodes and/or the management of recurrent episodes of genital herpes.

Another embodiment is a method for administering valacyclovir or a salt, ester, or prodrug thereof to an animal (preferably a mammal and more preferably a human) in need thereof, by administering a composition or dosage unit form(s) of the present invention to the animal. A preferred route of administration is oral.

Yet another embodiment is a method of treating conditions or disorders caused by a virus in an animal (preferably a mammal and more preferably a human) including the step of administering an effective amount of a composition or dosage unit form(s) of the present invention to an animal in need thereof. In other words, an effective amount of the delivery agent compound to facilitate the delivery of the valacyclovir or a salt, ester, or prodrug thereof and an effective amount (e.g., a therapeutically effective amount) of valacyclovir is administered.

Yet another embodiment is a method for treating conditions or disorders caused by a virus in an animal (preferably a mammal and more preferably a human) by administering to the animal a therapeutically effective amount of a composition or dosage unit form(s) of the present invention. Such conditions and disorders, include but are not limited to, those caused by viruses of the herpes family, for example, herpes simplex 1 and 2 viruses (HIV 1 and HSV 2), varicella zoster virus (VZV), cytomegalovirus (CMV), Epstein-Barr virus (EBV), and other herpes virus infections (e.g. feline herpes virus infections).

Another embodiment is a method of ti-eating virus infections, including herpes infections such as herpes simplex 1 and 2 viruses (HSV 1, HSV 2), varicella zoster virus (VZV), cylomegalovirus (CMV) and Epstein-Barr virus (EBV), and other herpes virus infections (e.g. feline herpes virus infections) in a human or non-human animal by administering an effective amount of a composition or dosage unit form of the present invention.

Yet another embodiment is a method of treating clinical conditions or symptoms which are caused by the viruses enumerated above, including herpetic karatitis, herpetic encaphalitis, cold sores and genital infections (caused by herpes simplex), chicken pox and shingles (caused by varicella zoster) CMV-pneumonia and retinitis, particularly in immunocompromised patients including renal and bone marrow transplant patients and patients with Acquired Immune Deficiency Syndrome (AIDS) by administering an effective amount of a composition or dosage unit form of the present invention. Epstein-Barr virus (EBV) causes infectious mononucleosis, and is also suggested as the causative agent of nasopharyngeal cancer, immunoblastic lymphoma, Burkitt's lymphoma and hairy leukoplakia.

Yet another embodiment is a method of treating viral infections in an animal (preferably a mammal and more preferably a human) in need thereof by administering to the animal a therapeutically effective amount of a composition or dosage unit form(s) of the present invention. The viral infections are those treatable with valacyclovir or a salt, ester, or prodrug thereof.

Yet another embodiment is a method for treatment of the initial episodes and/or the management of recurrent episodes of genital herpes in a human in need thereof by administering (preferably orally) an effective amount of the pharmaceutical composition of the present invention. Preferably for the treatment of initial genetic herpes, the pharmaceutical composition (which may contain, for example, 1000 mg of valacyclovir or a molar equivalent of a salt or prodrug thereof is administered two times daily. The treatment may be continued for 7 to 10 days. Preferably, the pharmaceutical composition provides bioavailability (i.e., AUC) substantially equivalent to or better than the current valacyclovir formulations marketed as Valtrex® when 1000 mg of valacyclovir is administered once or twice daily to a human to treat these conditions.

Yet another embodiment is a method for treatment of recurrent episodes and/or the management of recurrent episodes of genital herpes in a human in need thereof by administering (preferably orally) an effective amount of a pharmaceutical composition of the present invention to a human. In one embodiment for the treatment of initial genetic herpes, 500 mg twice a day for 3 to 5 days or 1000 mg once daily for 5 days of valacyclovir or a molar equivalent of a salt or prodrug thereof is administer ed. Preferably, the pharmaceutical composition provides bioavailability (i.e., AUC) substantially equivalent to or better than the current valacyclovir formulations marketed as Valtrex® (U.S. FDA NDA Nos. 20-550, 20-550/S21, 20-550/S19, 20-550/S10, 20-550/S13, 20-550/S16, 20-550/S12, 20-550/S 005) when 500 mg of vacyclovir is administered once or twice daily to treat these conditions.

In various embodiments, HIV-infected or other immunocompromised adults and adolescents may receive a 1000 mg dose or receive the 500 mg does 2 times daily of valacyclovir in the pharmaceutical compositions of the present invention for 5 to 10 days.

Yet another embodiment is a method for treatment of chronic suppression of genital herpes in a human in need thereof by administering (preferably orally) an effective amount of the pharmaceutical composition of the present invention. Preferably for the treatment of initial genetic herpes, the pharmaceutical composition (e.g., 500 mg to 1000 mg of valacyclovir or a molar equivalent of a salt or prodrug thereof) is administered 1 time daily. Preferably, the pharmaceutical composition provides bioavailability (i.e., AUC) substantially equivalent to or better than the current valacyclovir formulations marketed as Valtrex® (U.S. FDA NDA Nos. 20-550, 20-550/S21, 20-550/S19, 20-550/S10, 20-550/S13, 20-550/S16, 20-550/S12, 20-550/S005) when 500 mg of vacyclovir is administered once or twice daily. Accordingly, pharmaceutical compositions of the present invention can be used to treat any of these conditions.

Preferably for chronic suppressive therapy for recurrent genital herpes, the composition is administered once daily or less frequently. The treatment may be continued for up to 12 months, followed by reevaluation. Preferably, the composition provides bioavailability (i.e., AUC) substantially equivalent to or better than the current valacyclovir formulations marketed as Valtrex® (U.S. FDA NDA Nos. 20-550, 20-550/S21, 20-550/S19, 20-550/S10, 20-550/S13, 20-550/S6, 20-550/S12, 20-550/S 005) when:
(1) 1 g of valacyclovir is administered 1 times daily,
(2) 500 mg of valacyclovir is administered 1 times daily,

Treatment may be continued for up to 12 months, followed by re-evaluation.

Yet another embodiment is a method for treatment of herpes labialis (cold sore) in a human in need thereof by administering (preferably orally) an effective amount of the composition of the present invention. Preferably the composition is administered every 12 or more hours (for example, a treatment regimen including administering 2000 mg of valacyclovir every 12 hours). Preferably, the pharmaceutical composition provides bioavailability (i.e., AUC) substantially equivalent to or better than the current valacyclovir formulations marketed as Valtrex® (U.S. FDA NDA Nos. 20-550, 20-550/S21, 20-550/S19, 20-550/S10, 20-550/S13, 20-550/S16, 20-550/S12, 20-550/S 005) when 2000 mg is administered 1 times daily.

Yet another embodiment is a method for treatment of Herpes Zoster (Shingles, Zoster) in a human in need thereof by administering (preferably orally) an effective amount of the composition of the present invention. Preferably the composition (e.g., 1000 mg 3 times daily at about 8 hour intervals of valacyclovir or a molar equivalent of a salt or prodrug thereof) is administered every 8 hours for 7 days but no more than 14 days. Preferably, the pharmaceutical composition provides bioavailability (i.e., AUC) substantially equivalent to or better than the current valacyclovir formulations marketed as Valtrex® (U U.S. FDA NDA Nos. 20-550, 20-550/S21, 20-550/S19, 20-550/S10, 20-550/S13, 20-550/S86, 20-550/S82, 20-550/S 005) when 1000 mg of the current valacyclovir formulations marketed as Valtrex® (U.S. FDA NDA Nos. 20-550, 20-550/S21, 20-550/S19, 20-550/S10, 20-550/S13, 20-550/S16, 20-550/S82, 20-550/S 005) is administered 3 times daily.

Oral valacyclovir may be used for chronic suppression or maintenance prophylaxis (secondary prophylaxis) of HSV in HIV-infected adults or adolescents with frequent or severe recurrences. For example, a dosage of 500 mg 2 times daily may be administered for such treatment. Preferably the composition (for example, a treatment regimen including administering 500 mg of valacyclovir 2 times daily of valacyclovir or a molar equivalent of a salt or prodrug thereof) is administered twice daily. Preferably, the pharmaceutical composition provides bioavailability (i.e., AUC) substantially equivalent to or better than the current valacyclovir formulations marketed as Valtrex® (U.S. FDA NDA Nos. 20-550, 20-550/S21, 20-550/S19, 20-550/S10, 20-550/S13, 20-550/S16, 20-550/S12, 20-550/S 005) when 500 mg of the current acyclovir formulations marketed as Valtrex® (U.S. FDA NDA Nos. 20-550, 20-550/S21, 20-550/S19, 20-550/S10, 20-550/S13, 20-550/S16, 20-550/S12, 20-550/S 005) is administered 2 times daily.

Yet another embodiment is a method for treatment of CMV in a human in need thereof by administering (preferably orally) an effective amount of the composition of the present invention. Preferably the composition is administered 2 times daily (for example, a treatment regimen including administering 2000 mg of valacyclovir 2 times daily), Preferably, the pharmaceutical composition provides bioavailability (i.e., AUC) substantially equivalent to or better than the current valacyclovir formulations marketed as Valtrex® (U.S. FDA NDA Nos. 20-550, 20-550/S21, 20-550/S19, 20-550/S10, 20-550/S13, 20-550/S16, 20-550/S12, 20-550/S 005) when 2000 mg of the current acyclovir formulations marketed as Valtrex® (U.S. FDA NDA Nos. 20-550, 20-550/S21, 20-550/S19, 20-550/S10, 20-550/S13, 20-550/S16, 20-550/S12, 20-550/S 005) is administered 2 times daily to a human to treat these conditions.

Yet another embodiment is a method for reduction of the risk of transmission of genital herpes in a human in need thereof by administering (preferably orally) an effective amount of the composition of the present invention. Preferably the composition (e.g., 500 mg one times daily intervals of valacyclovir or a molar equivalent of a salt or prodrug thereof) is administered one time daily. Preferably, the pharmaceutical composition provides bioavailability (i.e., AUC) substantially equivalent to or better than the current valacyclovir formulations marketed as Valtrex® (U.S. FDA NDA Nos. 20-550, 20-550/S21, 20-550/S19, 20-550/S10, 20-550/S13, 20-550/S16, 20-550/S12, 20-550/S 005) when 2000 mg of the current acyclovir formulations marketed as Valtrex® (U.S. FDA NDA Nos. 20-550, 20-550/S21, 20-550/S19, 20-550/S10, 20-550/S13, 20-550/S16, 20-550/S12, 20-550/S 005) is administered one time daily.

Yet another embodiment is a method of preparing a composition of the present invention by mixing at least one delivery agent compound and valacyclovir or a salt, ester, or prodrug thereof.

Specific amounts of valacyclovir are in view of bioavailability of Valtrex® formulations available in the United States as of the filing date of this application. Based on the improved bioavailability of pharmaceutical compositions of the present invention, absolute dosage amounts of valacylovir may be scaled down, for example, by a factor of 0.4-0.9, or 0.6-0.7. Exact dosage levels can be determined by a person of ordinary skill in the art.

### EXAMPLES

The following examples illustrate the invention without limitation. All pails are given by weight unless otherwise indicated.

### Example 1

### Formulation # 1: Valacyclovir (API)/4-CNAB (350 MG/300 MG) Tablets

FORMULATION COMPONENTS

| | | | Calculated Amount |
|---|---|---|---|
| | Each tablet contains | | 20 tablets |
| Materials | (mg/tablet) | | (g) |
| Valacyclovir | 350.00 | | 7.00 |
| 4-CNAB (monosodium | 300.00 | | 6.00 |
| salt) | | | |
| Povidone* | 14.00 | | 0.28 |
| Pregelatinized | 82.00 | | 1.64 |
| Starch* | | | |
| Magnesium stearate* | 4.00 | | 0.08 |
| Total | 750.00 | mg | 15.00 |

*EXTRAGRANULAR

### Manufacturing Method

7.00 g of valacyclovir and 6.00 g of 4-CNAB were weighed and triturated to obtain a homogeneous mixture. The mixture was compacted using a roller compactor (Alexanderwerk) while maintaining the roller pressure of 40 bars and roller speed of 4 rpm. The compacted flake was sieved using a 35 mesh sieve (500 micron) to obtain valacyclovir/4-CNAB granules. 0.28 g of povidone and 1.64 g of pregelatinized starch were passed through a 35 mesh sieve and added to the valacyclovir/4CNAB granules and mixed for five minutes, and then 0.08 g magnesium stearate was passed through a 35 mesh sieve and added to the mixture and mixed for three minutes to obtain the final blend. The final blend was compressed using a Carver tablet press at a compression force of 1300 pounds to produce tables of 750 mg with a weight range of 727.5 mg-772.5 mg. The tablet disintegration time in water at 37±2° C. was not more than 10 minutes.

In-vivo Studies: The pharmacokinetic profiles of all the formulations described herein were carried out in a beagle model. Dog studies were conducted in accordance with the approved protocol by the Animal Care and Use Committee of Emisphere Technologies. Each beagle was administered orally the valacyclovir tablets formulated with a delivery agent compound. Dogs were fasted at least 8 hrs prior to dosing and were fed immediately after study. Fast not to exceed 24 hrs. Blood samples of about 0.5 ml volume was withdrawn from the Jugular vein before and after dosing. The time points for blood withdrawal were: −15, +5, 10, 20, 30, 40 min, 1, 1.5, 2, 3, 4, and 6 hr. post dose. The blood samples were put on ice immediately after collection then centrifuged for 10 minutes at 3000 RPM at approximately 4° C. (within 45 minutes of collection). The plasma samples were stored in −20° C. until time of analysis of acyclovir levels. Plasma acyclovir levels were analyzed by LC-MS method. The results were presented as individual acyclovir levels per dog or as the mean (+/−SE) from a group of four dogs.

TABLE 1
RESULTS OF FORMULATION #1

| Time (Mins) | (mcg/ml) | (mcg/ml) | (mcg/ml) | (mcg/ml) |
|---|---|---|---|---|
| (X) | Dog B | Dog C | Dog D | Dog E |
| −15 | 0 | 0 | 0 | 0 |
| 10 | 0 | 0 | 2433.5 | 1575.3 |
| 20 | 0 | 6878.9 | 7156.8 | 11375.6 |
| 30 | 0 | 7608.4 | 4155.2 | 14354.6 |
| 40 | 1611.5 | 18005.8 | 8586.3 | 12465.6 |
| 50 | 6393.9 | 22442.5 | 2964.9 | 13127 |
| 60 | 20154.3 | 17645.4 | 4538.1 | 9006.9 |
| 90 | 29592.2 | 22779 | 4370.9 | 8877.3 |
| Time (Mins) | 20245.7 | 14662 | 4304.1 | 6348.6 |
| 180 | 14140.8 | 14841.7 | 5103.2 | 4547.4 |
| 240 | 12174.7 | 11849.3 | 3291.8 | 3838.6 |
| 360 | 3432.9 | 2289.6 | 1592.4 | 3915 |
| 480 | 2311.6 | 985.6 | 1019.9 | 2141.1 |

TABLE 2
MEAN RESULTS OF FORMULATION #1

| | Plasma | |
|---|---|---|
| Time | acyclovir | |
| (min) | (mcg/ml) | SE (mcg/ml) |
| −15 | 0 | 0 |
| 10 | 1002.2 | 604.55721 |
| 20 | 6352.825 | 2354.24767 |
| 30 | 6529.55 | 3036.82792 |
| 40 | 10167.3 | 3445.11175 |
| 50 | 11232.08 | 4291.64391 |
| 60 | 12836.18 | 3653.82214 |
| 90 | 16404.85 | 5887.95345 |
| 120 | 11390.1 | 3705.34635 |
| 180 | 9658.275 | 2796.28737 |
| 240 | 7788.6 | 2441.8379 |
| 360 | 2807.475 | 529.33684 |
| 480 | 1614.55 | 355.00239 |

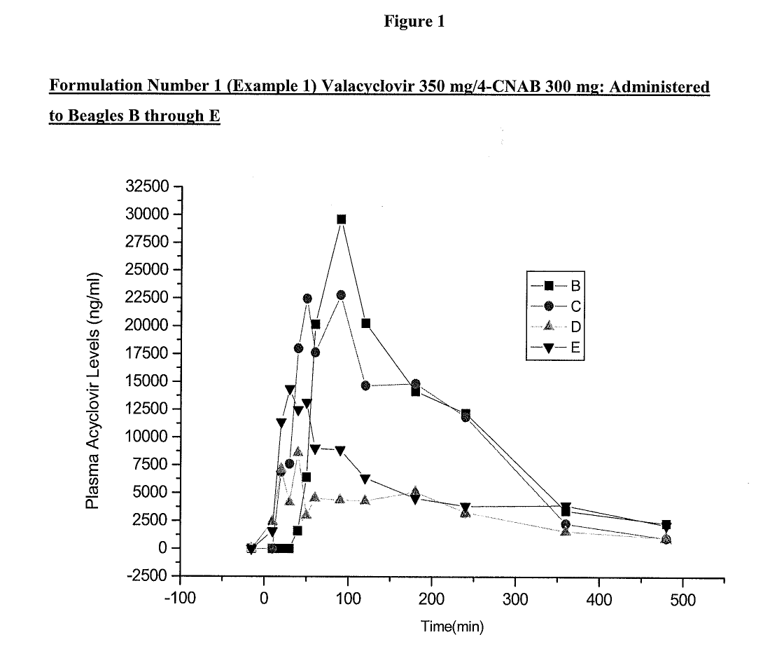

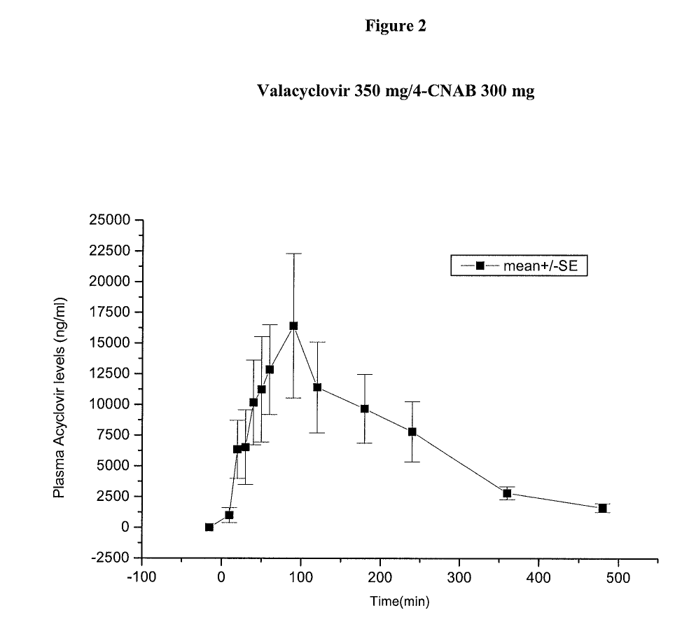

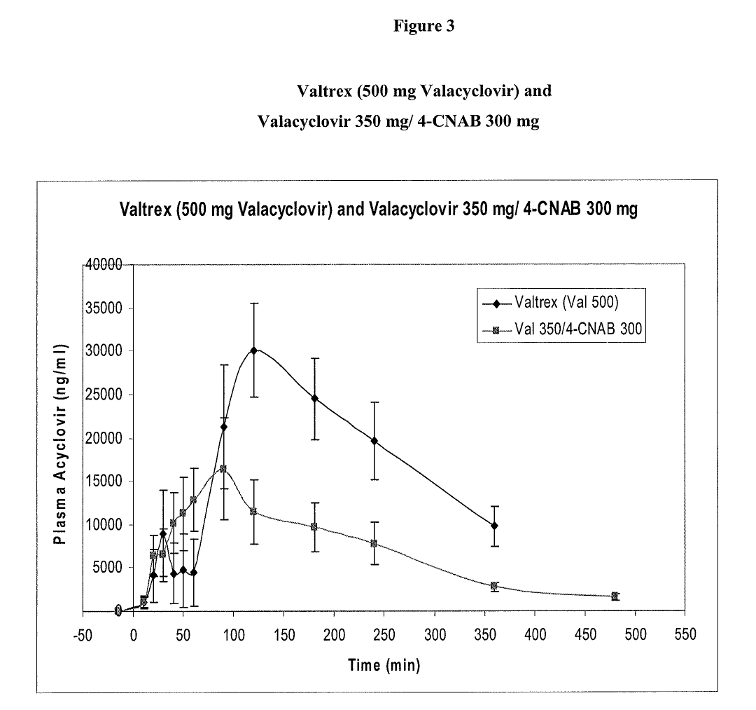

Results are also shown in FIGS. 1 and 2. FIG. 3 compares the results of the 350 mg formulation to a control of 500 mg of crushed Valtrex® powder, administered by the protocol described above.

### Example 2

### Formulation # 2: Valacyclovir (API)/4-CNAB (350 MG/300 MG) Tablets with Microcrystalline Cellulose

FORMULATION COMPONENTS

| | | | Calculated Amount |
|---|---|---|---|
| | Each tablet contains | | 20 tablets |
| Materials | (mg/tablet) | | (g) |
| Valacyclovir | 350.00 | | 7.00 |
| 4-CNAB (monosodium | 300.00 | | 6.00 |
| salt) | | | |
| Crospovidone | 20.00 | | 0.40 |
| Microcrystalline | 50.00 | | 1.00 |
| cellulose (Ceolus)* | | | |
| Povidone* | 18.00 | | 0.36 |
| Fumed silicon dioxide | 8.00 | | 0.16 |
| (Aerosil 200)* | | | |
| Magnesium stearate* | 4.00 | | 0.08 |
| Total | 750.00 | mg | 15.00 |

*EXTRAGRANULAR

### Manufacturing Method

7.00 g of valacyclovir, 6.00 g of 4-CNAB and 0.40 g of crospovidone were triturated to obtain a homogeneous mixture. The mixture was compacted using a roller compactor (Alexanderwerk) while maintaining the roller pressure of 40 bar and roller speed of 4 rpm. The compacted flake was hand sieved using a 35 mesh sieve (500 micron) to produce valacyclovir/4-CNAB granules. Then 1.00 g of microcrystalline cellulose (ceolus), 0.36 g of povidone and 0.16 g of fumed silicon dioxide (Aerosil 200) were passed through a 35 mesh sieve and added with the valacyclovir 4CNAB granules and mixed for five minutes. 0.08 g magnesium stearate was passed through a 35 mesh sieve and added to the mixture and then mixed for three minutes to obtain the final blend. The final blend was compressed using Carver tablet press at a force of 1300 pounds to produce tables of 750 mg with a weight range of 727.5 mg 772.5 mg. The tablet disintegration time in water at 37±2° C. was not more than 10 minutes.

Results of Formulation No 2

| Time | mcg/ml | mcg/ml | mcg/ml | mcg/ml |
|---|---|---|---|---|
| (min) | Dog B | Dog C | Dog D | Dog G |
| −15 | 0 | 0 | 0 | 0 |
| 10 | 666.5 | 1989.6 | 0 | 0 |
| 20 | 0 | 3220.1 | 4644.7 | 0 |
| 30 | 1289.7 | 9479.5 | 12811.4 | 0 |
| 40 | 605.6 | 7218.1 | 21722.7 | 1571 |
| 50 | 3102.3 | 12836.9 | 26829.1 | 15947.3 |
| 60 | 12604.7 | 17355.2 | 23707.8 | 23964.8 |
| 90 | 24009.9 | 13476.7 | 18650.6 | 17083.6 |
| 120 | 17991.1 | 14253 | 15423.3 | 17944.8 |
| 180 | 8049.7 | 11748.3 | 13305.7 | 10461.9 |
| 240 | 11681.9 | 7162.3 | 8745 | 13036.1 |
| 360 | 3797.5 | 3414.1 | 4864.4 | 1831.8 |
| 480 | 4706.2 | 1497.7 | 1829.5 | 2668.6 |

TABLE 10
Mean Results Formulation No 2

| Time | Mean Cp | |
|---|---|---|
| (min) | (Mcg/ml) | SE+/− |
| −15 | 0 | 0 |
| 10 | 664.025 | 468.9539 |
| 20 | 1966.2 | 1171.84 |
| 30 | 5895.15 | 3117.759 |
| 40 | 7779.35 | 4871.159 |
| 50 | 14678.9 | 4887.457 |
| 60 | 19408.13 | 2734.831 |
| 90 | 18305.2 | 2188.409 |
| 120 | 16403.05 | 934.5909 |
| 180 | 10891.4 | 1111.412 |
| 240 | 10156.33 | 1340.911 |
| 360 | 3476.95 | 628.3735 |
| 480 | 2675.5 | 720.3425 |

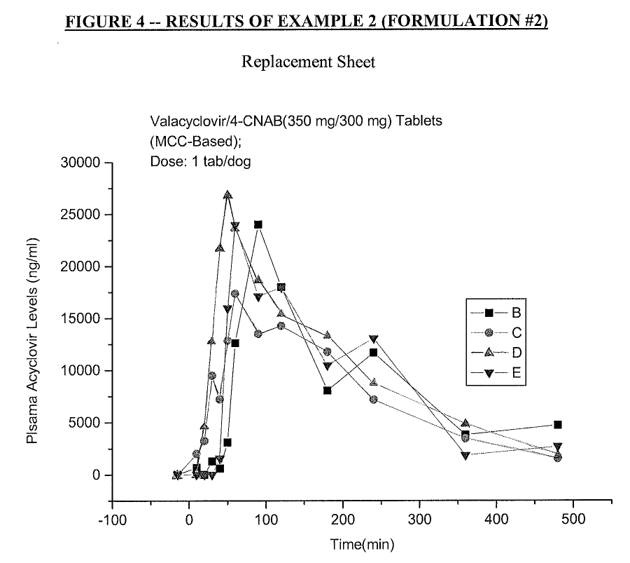

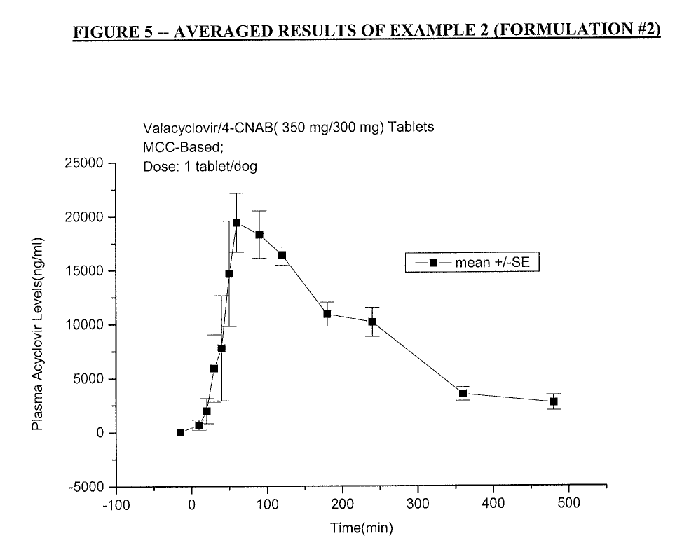

These results are also shown in FIGS. 4 and 5.

### Example 3

### Formulation # 3: Valacyclovir (API)/SNAC (350 MG/300 MG) Tablets

FORMULATION COMPONENTS

| | | | Calculated Amount |
|---|---|---|---|
| | Each tablet contains | | 20 tablets |
| Materials | (mg/tablet) | | (g) |
| Valacyclovir | 350.00 | | 7.00 |
| SNAC | 300.00 | | 6.00 |
| Povidone* | 14.00 | | 0.28 |
| Pregelatinized* | 82.00 | | 1.64 |
| starch | | | |
| Magnesium stearate* | 4.00 | | 0.08 |
| Total | 750.00 | mg | 15.00 |

*EXTRAGRANULAR

### Method of Manufacture

7.00 g of valacyclovir and 6.00 g of the monosodium salt of SNAC were weighed and triturated to obtain a homogeneous mixture. The mixture was compacted using a roller compactor (Alexanderwerk) while maintaining the roller pressure of 40 bars and roller speed of 4 rpm. The compacted flakes were sieved using 35 mesh (500 micron) sieve to produce granules. Then 0.28 g of povidone and 1.64 g of pregelatinized starch were passed through a 35 mesh screen and added to the valacyclovir/4-CNAB granules and mixed for five minutes. 0.08 g magnesium stearate was passed through a 35 mesh sieve and added to the mixture and mixed for three minutes to obtain the final blend. The final blend was compressed using a Carver tablet press while maintaining a pressure of 1300 pounds to produce tables of 750 mg with a weight range of 727.5 mg-772.5 mg. The tablet disintegration time in water at 37±2° C. was not more than 10 minutes.

### Example 4

### Formulation # 4: Valacyclovir (API)/SNAC (350 MG/300 MG) Tablets with Microcrystalline Cellulose

FORMULATION COMPONENTS

| | | | Calculated Amount |
|---|---|---|---|
| | Each tablet contains | | 20 tablets |
| Materials | (mg/tablet) | | (g) |
| Valacyclovir | 350.00 | | 7.00 |
| SNAC | 300.00 | | 6.00 |
| Crospovidone | 20.00 | | 0.40 |
| Microcrystalline | 50.00 | | 1.00 |
| cellulose (Ceolus)* | | | |
| Povidone* | 18.00 | | 0.36 |
| Fumed silicon dioxide | 8.00 | | 0.16 |
| (Aerosil 200)* | | | |
| Magnesium stearate* | 4.00 | | 0.08 |
| Total | 750.00 | mg | 15.00 |

*EXTRAGRANULAR

### Method of Manufacture

7.00 g of valacyclovir, 6.00 g of the monosodium salt of SNAG and 0.40 g of crosspovidone were triturated to obtain a homogeneous mixture. The mixture was compacted using a roller compactor (Alexanderwerk) while maintaining the roller pressure of 40 bar and roller speed of 4 rpm. The compacted flakes were hand sieved through a 35 mesh screen (500 micron). Then 1.00 g of microcrystalline cellulose (ceolus), 0.36 g of povidone and 0.16 g of fumed silicon dioxide (Aerosil 200) were passed through a 35 mesh screen and added with the valacyclovir/4-CNAB granules and mixed for five minutes. 0.08 g magnesium stearate was passed through a 35 mesh screen and added to the mixture and then mixed for three minutes to obtain the final blend. The final blend was compressed using a Carver press at a pressure of 1300 pounds to produce tablets of 750 mg with a weight range of 727.5 mg-772.5 mg. The tablet disintegration time in water at 37±2° C. was not more than 10 minutes.

### Example 5

### Prophetic

| | Each tablet contains | |
|---|---|---|
| Materials | (mg/tablet) | |
| Valacyclovir | 300 | |
| 4-CNAB (monosodium salt) | 300 | |
| *Polysorbate 80 (3.0% w/w) | 19.5 | |
| Povidone K90 (2.0% w/w) | 13.0 | |
| Anhydrous Emcompress ® (q.s) | 11.0 | |
| Magnesium stearate (1% w/w) | 6.5 | |
| Total | 650 | mg |

*Mixed with water to make the granulation fluid

### Method of Manufacture

The required amounts of valacyclovir and SNAC will be weighed out, screened through a 35 mesh screen and added to a suitably sized granulation bowl. The required amount of polysorbate 80 and Povidone will be weighed out and dissolved in water to make the granulation medium (the total volume of granulation fluid should be about 15% of the batch size by weight). The valacyclovir/4-CNAB blend will be granulated using the polysorbate 80/Povidone solution and granulation will be completed using water as required. The granules will then be dried in a vacuum oven at 50° C. until the moisture content is not more than 10% w/w. The dried granules will then be milled through a 35 mesh sieve and blended for 5 minutes with the required amount of Anhydrous Emcompress® in a V-blender or other suitable equipment to make a blend. The required amount of magnesium stearate will then be weighed out, screened through a 35 mesh screen and added to the blend and blended for 3 minutes. The resulting material will then be compressed in to tablets using a suitable tablet press.

### Example 6

### Prophetic

| | Each tablet contains | |
|---|---|---|
| Materials | (mg/tablet) | |
| Valacyclovir | 300 | |
| SNAC (monosodium salt) | 300 | |
| *Polysorbate 80 (3.0% w/w) | 19.5 | |
| Povidone K90 (2.0% w/w) | 13.0 | |
| Anhydrous Emcompress ® (q.s) | 11.0 | |
| Magnesium stearate (1% w/w) | 6.5 | |
| Total | 650 | mg |

*To be mixed with water to make the granulation fluid

### Method of Manufacture

The required amounts of valacyclovir and SNAC will be weighed out, screened through a 35 mesh screen and added to a suitably-sized granulation bowl. The required amount of polysorbate 80 and Povidone will be weighed out and dissolved in water to make the granulation medium (the total volume of granulation fluid should be about 15% of the batch size by weight). The valacyclovir/SNAC blend will be granulated using the polysorbate 80/Povidone solution and granulation will be completed using water as required. The granules will then be dried in a vacuum oven at 50° C. until the moisture content is not more than 10% w/w. The dried granules will then be milled through a 35 mesh sieve and blended for 5 minutes with the required amount of Anhydrous Emcompress® in a V-blender or other suitable equipment to make a blend. The required amount of magnesium stearate will then be weighed out, screened through a 35 mesh screen and added to the blend and blended for 3 minutes. The resulting material will be the compressed into tablets using a suitable tablet press.

### Example 7

### Prophetic

| | Each tablet contains | |
|---|---|---|
| Materials | (mg/tablet) | |
| Valacyclovir | 300 | |
| 4-CNAB (monosodium salt) | 300 | |
| Gelatin (5% w/w) | 32.5 | |
| Anhydrous Emcompress ® (q.s) | 11.0 | |
| Magnesium stearate (1% w/w) | 6.5 | |
| *Water | q.s | |
| Total | 650 | mg |

*Removed during drying

### Method of Manufacture

The required amounts of valacyclovir-4-CNAB and gelatin will be weighed out, screened through a 35 mesh screen and added to a suitably sized granulation bowl. The required amount of water will be weighed out for use as the granulation fluid. The volume of water should be about 15% of the batch size by weight. The valacyclovir, 4-CNAB, and gelatin blend will be granulated using the water as the granulating solution. The granules will then be dried in a vacuum oven at 50° C. until the moisture content is not more than 10% w/w. The dried granules will then be milled through a 35 mesh sieve and blended for 5 minutes with the required amount of Anhydrous Emcompress® in a V-blender blender or other suitable equipment to make a blend. The required amount of magnesium stearate will then be weighed out, screened through a 35 mesh screen and added to the blend and blended for 3 minutes. The resulting material will be the compressed in to tablets using a suitable tablets press.

### Example 8

### Solid Oral Delivery of Acyclovir in Beagles (Valacyclovir/SNAC (300 mg/300 mg) Tablets)

Method of Preparation: Tablets were prepared by the process of dry granulation. Crushed Valtrex® tablets were used in the study. Valtrex® is a commercial product of valacyclovir which is a prodrug of acyclovir. Each Valtrex® tablet used in the study contains 500 mg of valacyclovir. The average weight of a Valtrex® tablet is 728 mg. Therefore, 436 mg of crushed Valtrex® tablet will contain 300 mg of valacyclovir. To prepare Valtrex® tablets with a delivery agent compound, Valtrex® tablets were crushed in a mortar and a pestle. The crushed Valtrex® tablets were screened through a sieve #35 as well as all other ingredients of the formulation. To the screened Valtrex® powder, delivery agent, povidone, croscarmellose sodium and pregelatinized starch were added in the amounts shown below:

TABLE 3
ADDITIVES TO VALTREX POWDER

| Material | Amount (mg/tablet) |
|---|---|
| SNAC | 300 |
| Kollidon (Povidone K90) | 19.5 |
| Croscarmellose sodium | 13.0 |
| Pregelatinized starch | 11.0 |

The powder blends were mixed gently in a mortar and a pestle. Dry granules were obtained by compressing the powder blends into compacts which were later milled and screened through a sieve #35. Final tablets were obtained by compressing dry granules using a single-punch tablet press. The set of punches used was caplet shaped as fabricated by Natoli Engineering Company Inc. The punches and tablet die was lubricated with magnesium stearate (0.1%) during compression.

TABLE 4
Oral Delivery of Valcyclovir in Beagles
(Valacyclovir/SNAC (300 mg/300 mg) Tablets)

| | | | | Dog E |
|---|---|---|---|---|
| | Dog B | Dog C | Dog D | Plasma |
| | Plasma | Plasma | Plasma | Acyclovir |
| | Acyclovir | Acyclovir | Acyclovir | Con- |
| Time | Concentration | Concentration | Concentration | centration |
| (Mins) | (ng/ml) | (ng/ml) | (ng/ml) | (ng/ml) |
| −15 | 0 | 0 | 0 | 0 |
| 10 | 1711.6 | 0 | 0 | 0 |
| 20 | 15086.7 | 341.4 | 0 | 2748.2 |
| 30 | 30152.6 | 1494.1 | 1950.3 | 8022.3 |
| 40 | 34004.4 | 8718.1 | 10870.5 | 13480.6 |
| 50 | 35318.8 | 15993.8 | 13652.2 | 15337.8 |
| 60 | 32930.5 | 23527.1 | 14546.1 | 26305.6 |
| 90 | 22213.8 | 27190.6 | 17156.3 | 26090.3 |
| 120 | 20735.3 | 21869.2 | 14369.9 | 25970.6 |
| 180 | 14362.7 | 20193.4 | 12545.3 | 16430.3 |
| 240 | 9600 | 12299.2 | 9620.8 | 9518.5 |
| 360 | 4588.2 | 5729.3 | 3997.7 | 4941.7 |

TABLE 5
Oral Delivery of Valcyclovir in Dogs: Mean Plasma
Concentrations(Valacyclovir/SNAC (300 mg/300 mg) Tablets)

| | Mean Plasma Acyclovir | |
|---|---|---|
| Time (mins) | Concentration (ng/ml) | SE (+/−) |
| −15 | 0 | 0 |
| 10 | 427.9 | 427.9 |
| 20 | 4544.075 | 3567.0156 |
| 30 | 10404.825 | 6748.64836 |
| 40 | 16768.4 | 5827.24835 |
| 50 | 20075.65 | 5104.92453 |
| 60 | 24327.325 | 3810.52178 |
| 90 | 23162.75 | 2268.84832 |
| 120 | 20736.25 | 2401.60378 |
| 180 | 15882.925 | 1641.4079 |
| 240 | 10259.625 | 680.21657 |
| 360 | 4814.225 | 361.87158 |

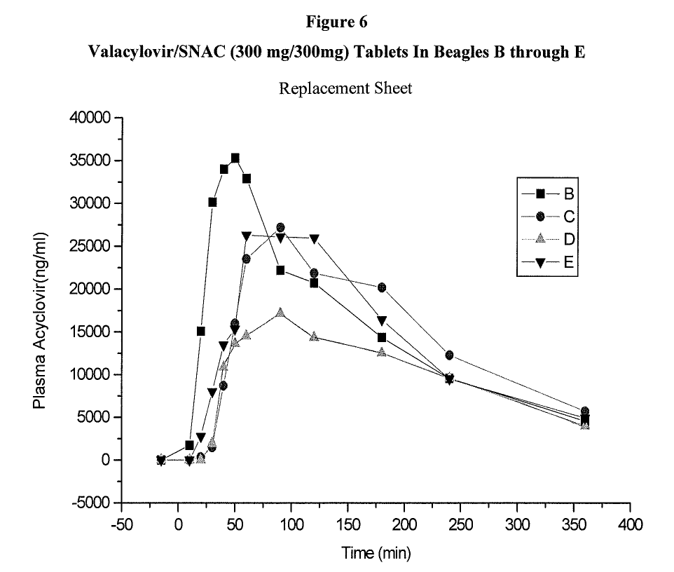

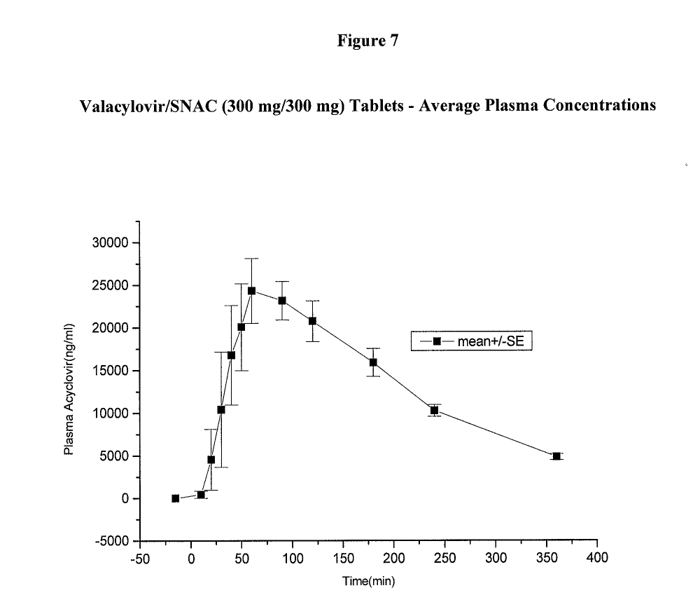

Results are also shown in FIGS. 6 and 7.

### Example 9

### Delivery, of Acyclovir in Dogs (Valaclovir/SNAD (300 ma/300 ma) Tablets)

Method of Preparation: Tablets were prepared as in example 8, except that the disodium salt of SNAD was used in place of the monosodium salt of SNAC as the delivery agent compound.

In-vivo Studies: Plasma acyclovir concentrations in beagles were obtained as in Example 8.

TABLE 6
The plasma Acyclovir concentration
(valaclovir/SNAD (300 mg/300 mg) Tablets)

| Time | Dog B | Dog C | Dog D | Dog E |
|---|---|---|---|---|
| −15 | 0 | 0 | 0 | 0 |
| 10 | 0 | 1231.1 | 573.2 | 0 |
| 20 | 0 | 4357.4 | 3953 | 0 |
| 30 | 0 | 7859.9 | 7726.8 | 4519.5 |
| 40 | 428.9 | 8312 | 11558.7 | 10534.8 |
| 50 | 1843.4 | 9038 | 11858.2 | 13879.3 |
| 60 | 15621 | 8957.2 | 13837.7 | 15097.1 |
| 90 | 18853.3 | 8721.4 | 13310.6 | 17984.9 |
| 120 | 14041.1 | 8329.5 | 11630.9 | 15099.3 |
| 180 | 8458.3 | 5577.8 | 9108.9 | 9590.1 |
| 240 | 4554.4 | 2987.5 | 5493.6 | 7298 |
| 360 | 0 | 1490.2 | 3245.8 | 3891.7 |

TABLE 7
The mean plasma Acyclovir concentration ± standard deviation (SD)):
Valaclovir/SNAD (300 mg/300 mg) Tablets)

| Time | Mean | SE |
|---|---|---|
| −15 | 0 | 0 |
| 10 | 451.075 | 293.01462 |
| 20 | 2077.6 | 1202.33997 |
| 30 | 5026.55 | 1844.86962 |
| 40 | 7708.6 | 2519.40469 |
| 50 | 9154.725 | 2631.5313 |
| 60 | 13378.25 | 1520.44813 |
| 90 | 14717.55 | 2340.1091 |
| 120 | 12275.2 | 1502.15441 |
| 180 | 8183.775 | 899.07712 |
| 240 | 5083.375 | 901.17308 |
| 360 | 2156.925 | 879.95931 |

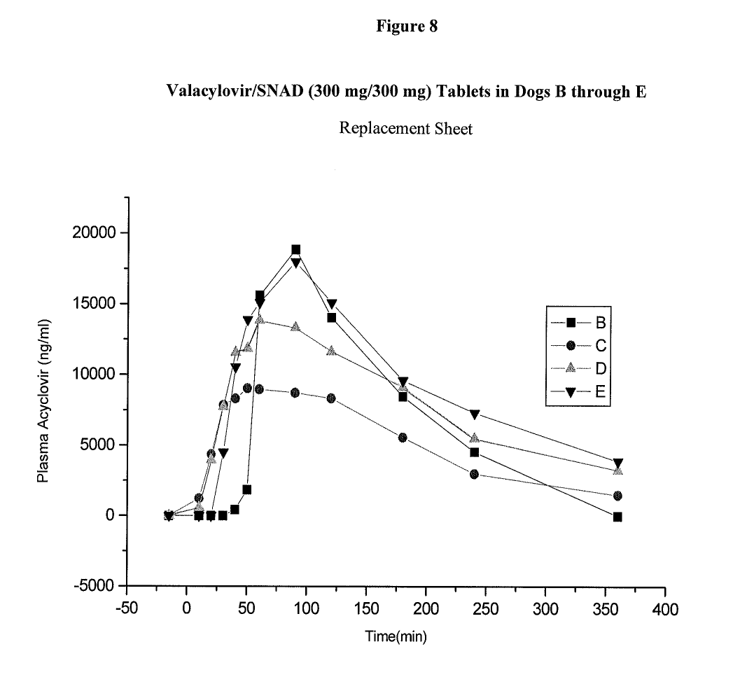

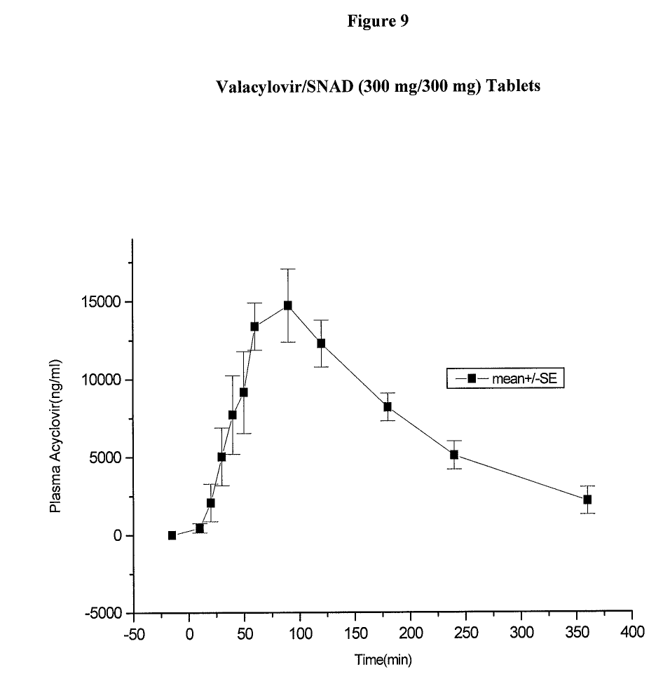

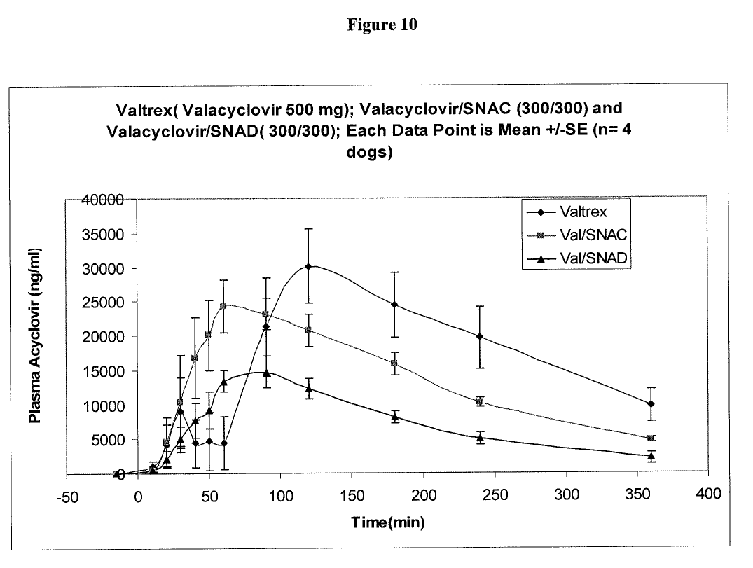

Results are also shown in FIGS. 8 and 9. FIG. 10 shows the results from Examples 8 and 9, against a 500 mg crushed Valtrex® control.

### Example 10

### Delivery of Acyclovir in Dogs (Valaclovir/4-CNAB (300 mg/300 mg) Tablets)

Method of Preparation: Tablets were prepared as in example 8, except that the monosodium salt of 4CNAB were used in place of the monosodium salt of SNAC as the delivery agent compound.

In-vivo Studies: Plasma acyclovir concentrations were measured in beagles as in Example 8.

TABLE 8
Delivery of Acyclovir in Beagles (Valaclovir/4-CNAB (300 mg/300 mg)
Tablets) Plasma Acyclovir Concentrations

| Time | Dog B | Dog C | Dog D | Dog E |
|---|---|---|---|---|
| −15 | 0 | 0 | 1280.5 | 2025 |
| 10 | 826.375 | 500.72636 | 5643.3 | 28008.1 |
| 20 | 11247.675 | 5655.23656 | 12499.6 | 28926.8 |
| 30 | 19635.05 | 3603.95784 | 17173.1 | 23822.1 |
| 40 | 25685.2 | 4087.87025 | 20967.9 | 26855.3 |
| 50 | 27992.45 | 2759.0054 | 26964.2 | 27235.6 |
| 60 | 27618.775 | 989.39039 | 28773.3 | 23445 |
| 90 | 25589.375 | 1184.88623 | 27124.2 | 22371.7 |
| 120 | 20228.9 | 2788.1371 | 17625.2 | 10291.3 |
| 180 | 14606.525 | 1581.99181 | 16530.8 | 11831.8 |
| 240 | 12470.55 | 1645.74976 | 6324.3 | 3429.5 |

TABLE 9
Delivery of Acyclovir in Beagles (Valaclovir/4-CNAB (300 mg/300 mg)
Tablets): Mean plasma Acyclovir concentration ± standard deviation.

| Time | | Standard |
|---|---|---|
| (mins) | Mean | Deviation |
| −15 | 0 | 0 |
| 10 | 826.375 | 500.72636 |
| 20 | 11247.675 | 5655.23656 |
| 30 | 19635.05 | 3603.95784 |
| 40 | 25685.2 | 4087.87025 |
| 50 | 27992.45 | 2759.0054 |
| 60 | 27618.775 | 989.39039 |
| 90 | 25589.375 | 1184.88623 |
| 120 | 20228.9 | 2788.1371 |
| 180 | 14606.525 | 1581.99181 |
| 240 | 12470.55 | 1645.74976 |
| 360 | 5644.225 | 1400.3211 |

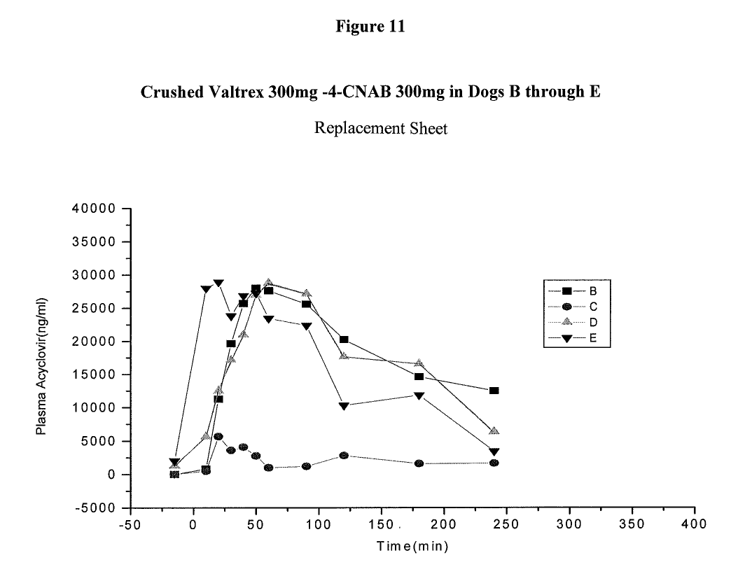

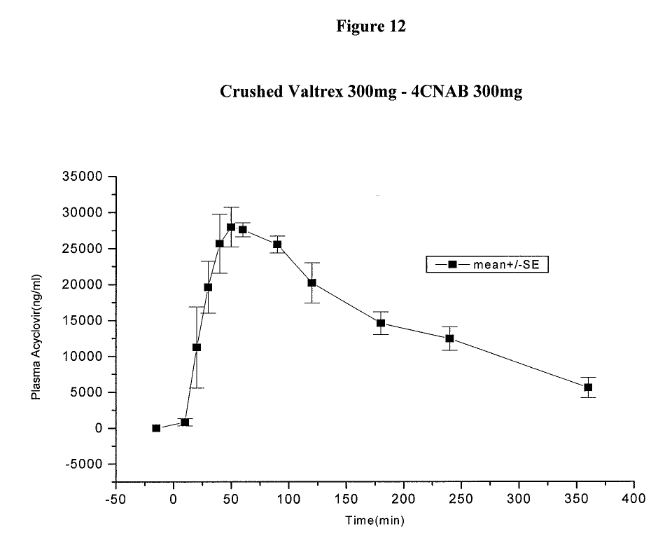

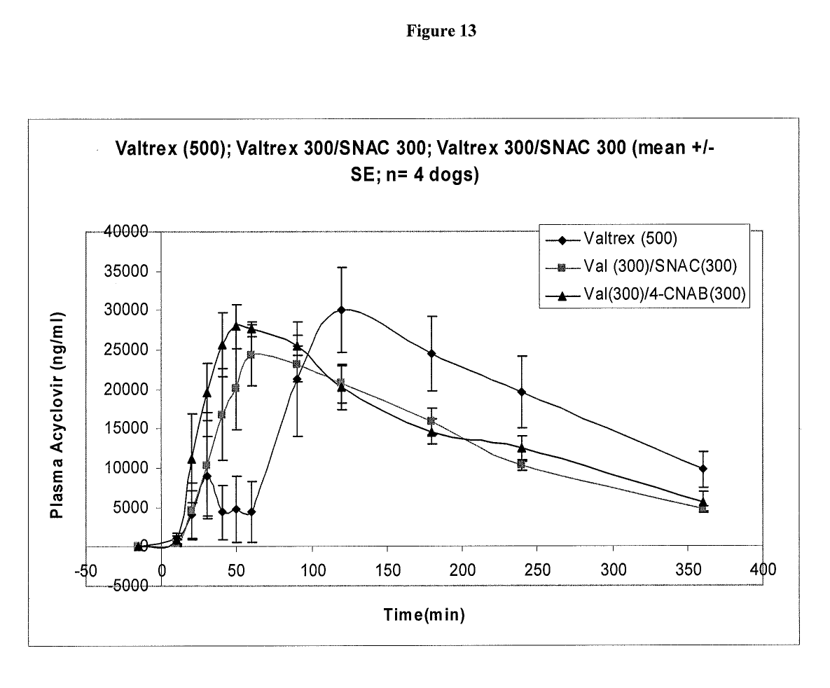

Results are also shown in FIGS. 11 and 12. Results from Example 8 and 10 against a crushed Valtrex® control (500 mg valacyclovir) are shown in FIG. 13.

### Example 11

### Delivery of Acyclovir in Beagles (Valaclovir/SNAC (500 mg/10 mg) Tablets)

Valtrex® tablets (containing 500 mg of valacyclovir) were ground into a powder and 100 mg of the monosodium salt of SNAC was added. The resulting binary mixture was formed into a tablet and administered to four beagles by the protocol described in Example 1.

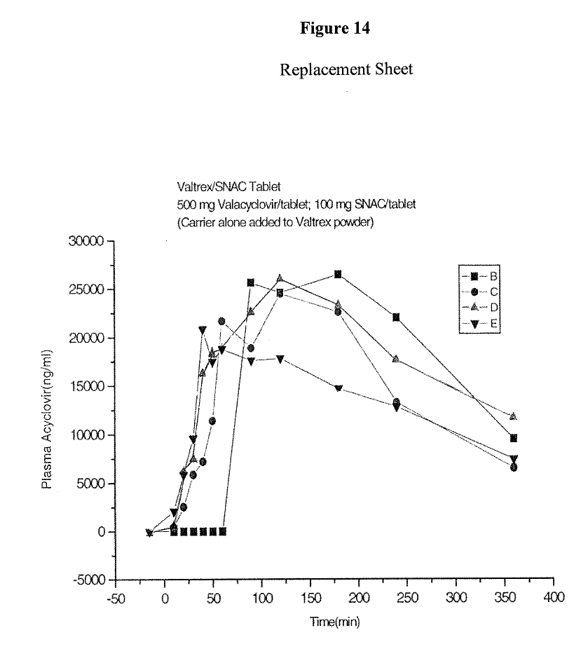

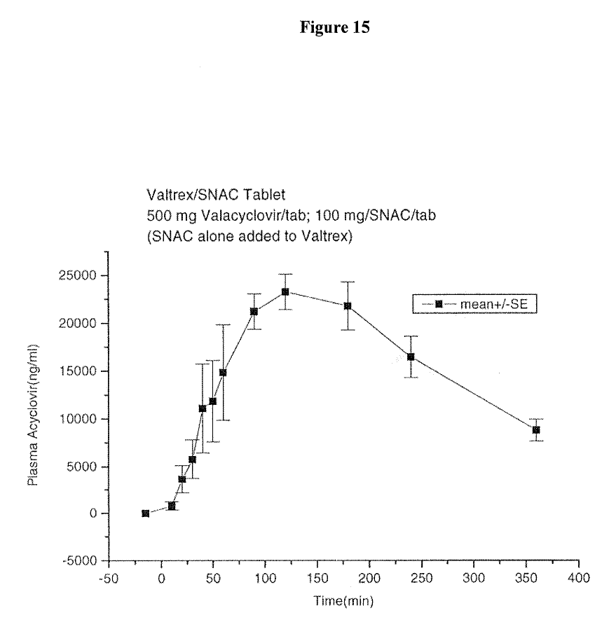

The results are shown in FIGS. 14 and 15.

### Example 12

### Delivery of Acyclovir in Beagles (Valaclovir/SNAC (500 mg/100 mg) Tablets)

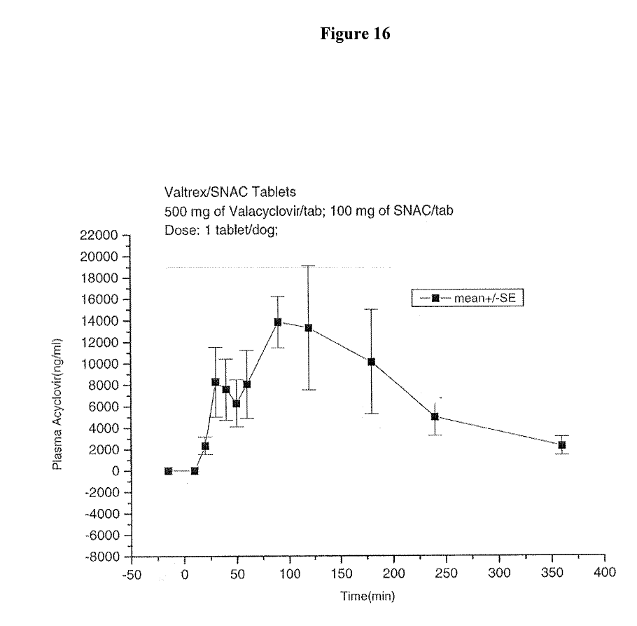

Tablets were prepared as in Example 11, except that croscarmellose sodium (10 mg/tablet) and pregelatinized starch (40 mg/tablet) were added to the mass prior to tableting. The results are shown in FIG. 16.

### Example 13

### Delivery of Acyclovir in Beagles (Valaclovir/SNAC (500 mg/200 mg) Tablets)

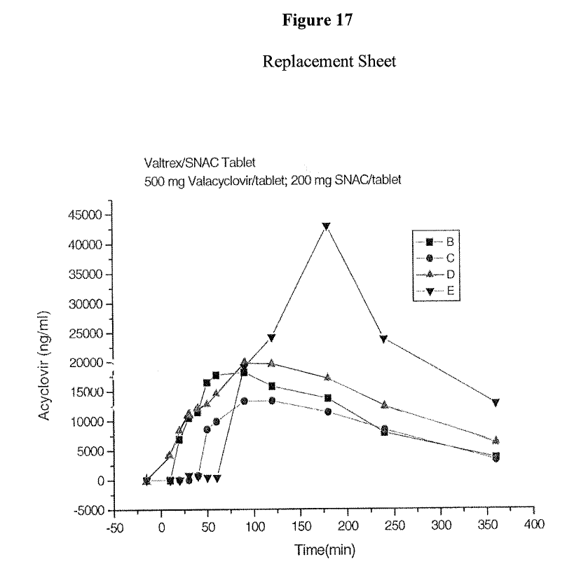

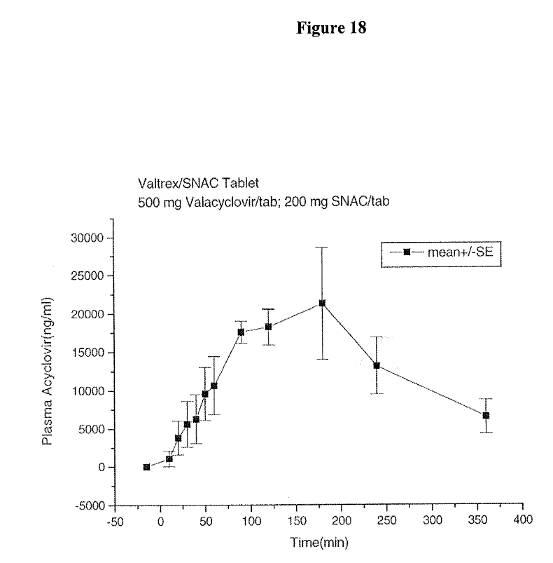

Tablets were prepared as in Example 12, except that 200 mg of the monosodium salt of SNAC was added instead of 100 mg. The results are shown in FIGS. 17 and 18.

The above-mentioned patents, applications, test methods, and publications are hereby incorporated by reference in their entirety.

Many variations of the present invention will suggest themselves to those skilled in the art in light of the above detailed description. All such obvious variations are within the fully intended scope of the appended claims.

1. A pharmaceutical composition comprising (a) valacyclovir or a salt thereof, and (b) at least one delivery agent selected from the following compounds, and pharmaceutically acceptable salts thereof:
(Genus a)2-HO—Ar—C(O)—NR⁸—R⁷—COOH Formula (1)
wherein
Ar is phenyl or naphthyl, optionally substituted with OH, halogen, C₁-C₄ alkyl, C₁-C₄ alkenyl, C₁-C₄ alkoxy or C₁-C₄ haloalkoxy;
R⁷ is C₄-C₂₀ alkyl, C₄-C₂₀ alkenyl, phenyl, naphthyl (C₁-C₁₀ alkyl)phenyl, (C₁-C₁₀ alkenyl)phenyl, (C₁-C₁₀ alkyl) naphthyl, (C₁-C₁₀ alkenyl) naphthyl, phenyl(C₁-C₁₀ alkyl), phenyl(C₁-C₁₀ alkenyl), naphthyl(C₁-C₁₀ alkyl), or naphthyl(C₁-C₁₀ alkenyl);
R⁸ is hydrogen, C₁ to C₄ alkyl, C₂ to C₄ alkenyl, C₁ to C₄ alkoxy, C₁-C₄ or haloalkoxy;
R⁷ is optionally substituted with C₁ to C₄ alkyl, C₂ to C₄ alkenyl, C₁ to C₄ alkoxy, C₁-C₄ haloalkoxy, —OH, —SH, and —CO₂R⁹ or any combination thereof;
R⁹ is hydrogen, C₁ to C₄ alkyl or C₂ to C₄ alkenyl; and
R⁷ is optionally interrupted by oxygen, nitrogen, sulfur or any combination thereof; with the proviso that the compounds are not substituted with an amino group in the position alpha to the acid group;
(Genus b)2-OH—Ar—C(O)—NH—R¹—R² Formula (2)
wherein
Ar is phenyl or naphthyl;
Ar is optionally substituted with C₁-C₄ alkyl, C₁-C₄ alkoxy, C₂-C₄ alkenyl, C₂-C₄ alkynyl, aryl, aryloxy, a heterocyclic ring, C₅-C₇ carbocylic ring, halogen, —OH, —SH, CO₂R⁶, —NR⁷R⁸, or —N⁺R⁷R⁸R⁹Y⁻;
(a) R¹ is C₁-C₁₆ alkylene, C₂-C₁₆ alkenylene, C₂-C₁₆alkynylene, C₆-C₁₆ arylene, (C₁-C₁₆alkyl)arylene, or aryl (C₁-C₁₆ alkylene);
R² is —NR³R⁴ or —N⁺R³R⁴R⁵Y⁻;
R³ and R⁴ are independently hydrogen; oxygen; hydroxy; substituted or unsubstituted C₁-C₁₆ alkyl; substituted or unsubstituted C₂-C₁₆ alkenyl; substituted or unsubstituted C₂-C₁₆ alkynyl; substituted or unsubstituted aryl; substituted or unsubstituted alkylcarbolnyl; substituted or unsubstituted arylcarbonyl; substituted or unsubstituted alkanesulfinyl; substituted or unsubstituted arylsulfinyl; substituted or unsubstituted alkanesulfonyl; substituted or unsubstituted arylsulfonyl; substituted or unsubstituted alkoxycarbonyl; substituted or unsubstituted aryloxycarbonyl;
R⁵ is independently hydrogen; substituted or unsubstituted C₁-C₁₆ alkyl; substituted or unsubstituted C₂-C₁₆ alkenyl; substituted or unsubstituted C₂-C₁₆ alkynyl; substituted or unsubstituted aryl; substituted or unsubstituted alkylcarbonyl; substituted or unsubstituted arylcarbonyl; substituted or unsubstituted alkanesulfinyl; substituted or unsubstituted arylsulfinyl; substituted or unsubstituted alkanesulfonyl; substituted or unsubstituted arylsulfonyl; substituted or unsubstituted alkoxycarbonyl; substituted or unsubstituted aryloxycarbonyl;
(b) R¹, R², and R⁵ are as defined above; and
R³ and R⁴ are combined to form a 5, 6 or 7-membered heterocyclic ring; or 5, 6 or 7-membered heterocyclic ring substituted with a C₁-C₆ alkyl, C₁-C₆ alkoxy, aryl, aryloxy, oxo group or carbocyclic ring; or
(c) R² and R⁵ are as defined above; and
R¹ and R³ are combined to form a 5, 6 or 7-membered heterocyclic ring; or 5, 6 or 7-membered heterocyclic ring substituted with a C₁-C₆ alkyl, alkoxy, aryl, aryloxy, or oxo group or carbocyclic ring;
R⁴ is hydrogen; oxygen; hydroxy; substituted or unsubstituted C₁-C₁₆ alkyl; substituted or unsubstituted C₂-C₁₆ alkenyl; substituted or unsubstituted C₂-C₁₆ alkynyl; substituted or unsubstituted aryl; substituted or unsubstituted alkylcarbonyl; substituted or unsubstituted arylcarbonyl; substituted or unsubstituted alkanesulfinyl; substituted or unsubstituted arylsulfinyl; substituted or unsubstituted alkanesulfonyl; substituted or unsubstituted arylsulfonyl; substituted or unsubstituted alkoxycarbonyl; substituted or unsubstituted aryloxycarbonyl;
R⁶ is hydrogen; C₁-C₄ alkyl; C₁-C₄ alkyl substituted halogen or —OH; C₂-C₄ alkenyl; or C₂-C₄ alkenyl substituted halogen or —OH;
R⁷, R⁸, and R⁹ are independently hydrogen; oxygen; C₁-C₄ alkyl; C₁-C₄ alkyl substituted with halogen or —OH; C₂-C₄ alkenyl; or C₂-C₄ alkenyl substituted with halogen or —OH; and
Y is halogen, hydroxide, sulfate, nitrate, phosphate, alkoxy, perchlorate, tetrafluoroborate, or carboxylate; 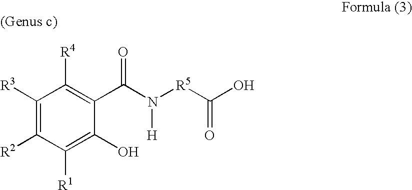
wherein
R¹, R², R³, and R⁴ are independently hydrogen, —OH, —NR⁶R⁷, halogen, C₁-C₄ alkyl, or C₁-C₄ alkoxy;
R⁵ is a substituted or unsubstituted C₂-C₁₆ alkylene, substituted or unsubstituted C₂-C₁₆ alkenylene, substituted or unsubstituted C₁-C₁₂ alkyl(arylene), or substituted or unsubstituted aryl(C₁-C₁₂ alkylene); and
R⁶ and R⁷ are independently hydrogen, oxygen, or C₁-C₄ alkyl; 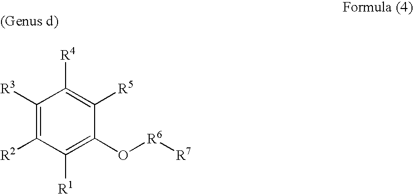
wherein
(a) R¹, R², R³, and R⁴ are independently H, —OH, halogen, C₁-C₄ alkyl, C₁-C₄ alkenyl, C₁-C₄ alkoxy, —C(O)R⁸, —NO₂, —NR⁹R¹⁰, or —N⁺R⁹R¹⁰R¹¹(Y⁻);
R⁸ is hydrogen, —OH, C₁-C₆ alkyl, C₁-C₄ alkyl substituted with halogen or —OH, C₂-C₄ alkenyl unsubstituted or substituted with halogen or OH, or —NR¹⁴R¹⁵;
R⁹, R¹⁰, and R¹¹ are independently hydrogen, oxygen, C₁-C₄ alkyl unsubstituted or substituted with halogen or —OH, C₂-C₄ alkenyl unsubstituted or substituted with halogen or —OH;
Y is halide, hydroxide, sulfate, nitrate, phosphate, alkoxy, perchlorate, tetrafluoroborate, carboxylate, mesylate, fumerate, malonate, succinate, tartrate, acetate, gluconate, maleate;
R⁵ is H, —OH, —NO₂, halogen, CF₃, —NR¹⁴R¹⁵, —N⁺R¹⁴R¹⁵R¹⁶(Y⁻), amide, C₁-C₁₂ alkoxy, C₁-C₁₂ alkyl, C₂-C₁₂ alkenyl, carbamate, carbonate, urea, or C(O)R²²; R⁵ is optionally substituted with halogen, —OH, —SH, or —COOH; R⁵ is optionally interrupted by O, N, S, or —C(O)—;
R¹⁴, R¹⁵ and R¹⁶ are independently H or C₁-C₁₀ alkyl;
R²² is H, C₁-C₆ alkyl, —OH, —NR¹⁴R¹⁵;
R⁶ is substituted or unsubstituted C₁-C₁₆ alkylene, C₂-C₁₆ alkenylene, C₂-C₁₆ alkynylene, C₅-C₁₆ arylene, (C₁-C₁₆ alkyl) arylene or aryl(C₁-C₁₆ alkylene); R⁶ is optionally substituted with C₁-C₇ alkyl or C₁-C₇ cycloalkyl;
R⁷ is —N¹⁸R¹⁹R¹⁹ or —N⁺R¹⁸R¹⁹R²⁰Y⁻;
R¹⁸ and R¹⁹ are independently hydrogen, oxygen, hydroxy, substituted or unsubstituted C₁-C₁₆(alkyl, substituted or unsubstituted C₂-C₁₆ alkenyl, substituted or unsubstituted C₂-C₁₆ alkynyl, substituted or unsubstituted aryl, substituted or unsubstituted alkylcarbonyl (e.g. substituted or unsubstituted (C₁₋₆ alkyl)carbonyl), substituted or unsubstituted arylcarbonyl, substituted or unsubstituted alkanesulfinyl (e.g. substituted or unsubstituted (C₁₋₆ alkane)sulfinyl), substituted or unsubstituted arylsulfinyl, substituted or unsubstituted alkanesulfonyl (e.g. substituted or unsubstituted (C₁₋₆ alkane)sulfonyl), substituted or unsubstituted arylsulfonyl, substituted or unsubstituted alkoxycarbonyl (e.g. substituted or unsubstituted (C₁₋₆ alkoxy)carbonyl), or substituted or unsubstituted aryloxycarbonyl, or substituted or unsubstituted C₅-C₇ heterocyclic ring (i.e., 5, 6, or 7-membered heterocyclic ring), wherein the substitutions may be halogen or —OH; and
R²⁰ is independently hydrogen, substituted or unsubstituted C₁-C₁₆ alkyl, substituted or unsubstituted C₂-C₁₆ alkenyl, substituted or unsubstituted C₂-C₁₆ alkynyl, substituted or unsubstituted aryl, substituted or unsubstituted alkylcarbonyl (e.g. substituted or unsubstituted (C₁₋₆ alkyl)carbonyl), substituted or unsubstituted arylcarbonyl, substituted or unsubstituted alkanesulfinyl (e.g. substituted or unsubstituted (C₁₋₆ alkane)sulfinyl), substituted or unsubstituted arylsulfinyl, substituted or unsubstituted alkanesulfonyl (e.g. substituted or unsubstituted (C₁₋₆ alkane)sulfonyl), substituted or unsubstituted arylsulfonyl, substituted or unsubstituted alkoxycarbonyl (e.g. substituted or unsubstituted (C₁₋₆ alkoxy)carbonyl), or substituted or unsubstituted aryloxycarbonyl; or
(b) R¹-R¹⁶ and R²⁰ are as defined above; and
R¹⁸ and R¹⁹ combine to form a 5, 6, or 7-membered heterocyclic ring optionally interrupted with an oxo group and unsubstituted or substituted with C₁-C₆ alkyl, C₁-C₆ alkoxy, aryl, aryloxy, or carbocyclic ring; 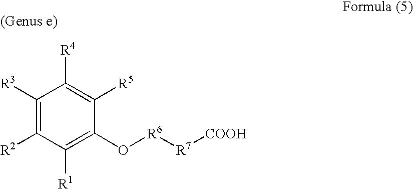
wherein
R¹, R², R³, and R⁴ are independently H, —OH, halogen, C₁-C₄ alkyl, C₂-C₄ alkenyl, C₁-C₄ alkoxy, —C(O)R⁸, —NO₂, —NR⁹R¹⁰, or —N⁺R⁹R¹⁰R¹¹(R¹²);
R⁵ is H, —OH, —NO₂, halogen, —CF₃, —NR¹⁴R¹⁵, —N⁺R¹⁴R¹⁵R¹⁶ (R¹³)⁻, amide, C₁-C₁₂ alkoxy, C₁-C₁₂ alkyl, C₂-C₁₂ alkenyl, carbamate, carbonate, urea, or —C(O)R¹⁸;
R⁵ is optionally substituted with halogen, —OH, —S11, or —COOH;
R⁵ is optionally interrupted by O, N, S, or —C(O)—;
R⁶ is a C₁-C₁₂ alkylene, C₂-C₁₂ alkenylene, or arylene;
R⁶ is optionally substituted with a C₁-C₄ alkyl, C₂-C₄ alkenyl, C₁-C₄ alkoxy, —OH, —SH, halogen, —NH₂, or —CO₂R⁸;
R⁶ is optionally interrupted by O or N;
R⁷ is a bond or arylene;
R⁷ is optionally substituted with —OH, halogen, —C(O)CH₃, —NR¹⁰R¹¹, or —N⁺R¹⁰R¹¹R¹² (R¹³)⁻;
R⁸ is H, C₁-C₄ alkyl, C₂-C₄ alkenyl, or —NH₂;
R⁹, R¹⁰, R¹¹, and R¹² independently H or C₁-C₁₀ alkyl;
R¹³ is a halide, hydroxide, sulfate, tetrafluoroborate, or phosphate; and
R¹⁴, R¹¹ and R¹⁶ are independently H, C₁-C₁₀ alkyl, C₁-C₁₀ alkyl substituted with —COOH, C₂-C₁₂ alkenyl, C₂-C₁₂ alkenyl substituted with —COOH, —C(O)R¹⁷;
R¹⁷ is —OH, C₁-C₁₀ alkyl, or C₂-C₁₂ alkenyl; and
R¹⁸ is H, C₁-C₆ alkyl, —OH, —NR¹⁴R¹⁵, or N⁺R¹⁴R¹⁵R¹⁶(R¹³); and 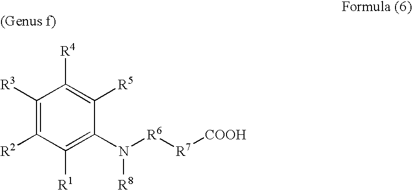
wherein
R¹, R², R³, and R⁴ are independently H, —OH, halogen, —OCH₃, —NR¹⁰R¹¹ or —N+R¹⁰R¹¹R¹² (R¹³)⁻;
R⁵ is H, —OH, —NO₂, —NR¹⁴R¹⁵, —N⁺R¹⁴R¹⁵R¹⁶ (R¹³)⁻, amide, C₁-C₁₂ alkoxy, C₁-C₁₂ alkyl, C₂-C₁₂ alkenyl, carbamate, carbonate, urea, or —C(C)R¹⁸;
R⁵ is optionally substituted with —OH, —SH, or —COOH;
R⁵ is optionally interrupted by O, N, S, or —C(O)—;
R⁶ is a C₁-C₁₂ alkylene, C₁-C₁₂ alkenylene, or arylene;
R⁶ is optionally substituted with a C₁-C₄ alkyl, C₂-C₄ alkenyl, C₁-C₄ alkoxy, —OH, —SH, halogen, —NH₂, or —CO₂R⁹;
R⁶ is optionally interrupted by C or N;
R⁷ is a bond or arylene;
R⁷ is optionally substituted with —OH, halogen, —C(O)CH₃, —NR¹⁰R¹¹ or —N⁺R¹⁰R¹¹R¹²(R¹³);
R⁸ is H or C₁-C₄ alkyl;
R⁹ is 1, C₁-C₄ alkyl, or C₂-C₄ alkenyl;
R¹⁰, R¹¹, and R¹² are independently H or C₁-C₁₀ alkyl;
R¹³ is a halide, hydroxide, sulfate, tetrafluoroborate, or phosphate;
R¹⁴, R¹⁵, and R¹⁶ are independently H, C₁-C₁₀ alkyl, C₂-C₁₂ alkenyl, O, or —C(O)R¹⁷;
R¹⁷ is —OH, C₁-C₁₀ alkyl, or C₂-C₁₂ alkenyl; and
R¹⁸ is —OH, C₁-C₆ alkyl, —NR¹⁴R¹⁵, —N⁺R¹⁴R¹⁵R¹⁶(R¹³)⁻; and 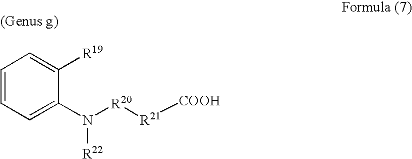
wherein
R¹⁹ is —NO₂ or —C(O)R²³;
R²⁰ is a C₁-C₁₂ alkylene or C₁-C₁₂ alkenylene;
R²¹ is a bond or arylene;
R²² is H or C₁-C₄ alkyl; and
R²³ is —OH, C₁-C₆ alkyl, or —NH₂.

2. A pharmaceutical composition of claim 1, wherein the delivery agent is selected from the group consisting of SNAC, SNAD, 4CNAB, and pharmaceutically acceptable salts thereof.

3. The pharmaceutical composition of claim 1 wherein the delivery agent is SNAC or a pharmaceutically acceptable salt thereof.

4. The pharmaceutical composition of claim 1 wherein the delivery agent is wherein the delivery agent is SNAD or a pharmaceutically acceptable salt thereof.

5. The pharmaceutical composition of claim 1 wherein the delivery agent is 4-CNAB or a pharmaceutically acceptable salt thereof.

6. The pharmaceutical composition of any one of claims 1-5, further comprising at least one component selected from the group consisting of povidone, starch, pregelatinized starch, magnesium stearate, crospovidone, cellulose, microcrystalline cellulose, fumed silicon dioxide, polysorbate, calcium phosphate, dibasic calcium phosphate, gelatin, and croscarmellose sodium.

7. The pharmaceutical composition of any one of claims 1-6 comprising from about 200 mg to about 400 mg of valacyclovir or a salt thereof.

8. The pharmaceutical composition of any one of claims 1-6 comprising from about 300 mg to about 350 mg of valacyclovir or a salt thereof.

9. The pharmaceutical composition of any claims 1-8, wherein the pharmaceutical composition provides acyclovir bioavailability substantially equivalent to 500 mg valacyclovir formulations marketed as Valtrex® under U.S. FDA NDA No. (U.S. FDA NDA Nos. 20-550, 20-550/S21, 20-550/S19, 20-550/S10, 20-550/S13, 20-550/S16, 20-550/S12, 20-550/S 005) when administered to a human.

10. A dosage unit form comprising:
(A) the pharmaceutical compositions of any one of the preceding claims; and
(B) (a) an excipient,
(b) a diluent,
(c) a disintegrant,
(d) a lubricant,
(e) a plasticizer,
(f) a colorant,
(g) a dosing vehicle, or
(h) any combination thereof.

11. The dosage unit form of claim 10, wherein the dosage unit form is in the form of a tablet, a capsule, a particle, a powder, a sachet, or a liquid.

12. The dosage unit four of claim 10, wherein the dosing vehicle is a liquid selected from the group consisting of water, aqueous propylene glycol, phosphate buffer, 1,2-propane diol, ethanol, and any combination thereof.

13. A method for administering an effective amount of valacyclovir a patient in need of thereof, comprising the step of orally administering the pharmaceutical composition of any one of claims 1-12.

14. A method of treating a viral infection in a patient in need thereof, comprising the step of administering to the patient an effective amount of the pharmaceutical composition of any one of claims 1-12.

15. A method of treating a condition or disorder caused by a virus in a patient in need thereof, comprising the step of administering an animal an effective amount of the pharmaceutical composition of any one of claims 1-12.

16. The method of claim 15, wherein the condition or disorder is caused by a virus selected from the group consisting of herpes simplex 1, herpes simplex 2, varicella zoster virus, cytomegalovirus and Epstein-Barr virus.

17. A method of improving the bioavailability of valacyclovir in an animal in need thereof, the method comprising the step of administering the composition of any one of claims 1-12.

18. A method of preparing a valacyclovir pharmaceutical composition comprising the step of mixing at least one delivery agent compound of claim 1 and valacyclovir or a salt or prodrug thereof.

19. A kit comprising any of the pharmaceutical compositions of claims 1-12.
